(19) United States
(12) Patent Application Publication
(10) Pub. No.: US 2026/0166031 A1
(43) Pub. Date: Jun. 18, 2026

(54) Methods of Treating Lipedema and Lymphatic Disease of the Subcutaneous Tissue

(71) Applicants: Celltaxis, LLC, Atlanta, GA (US); The Board of Trustees of the Leland Stanford Junior University, Stanford, CA (US)

(72) Inventors: Stanley Glenn ROCKSON, Palo Alto, CA (US); Robert Carson SIBLEY, Palo Alto, CA (US); Eric B. SPRINGMAN, Punta Gorda, FL (US); Eileen HEFFERNAN, Osterville, MA (US)

(21) Appl. No.: 19/413,887

(22) Filed: Dec. 9, 2025

Related U.S. Application Data
(63) Continuation of application No. PCT/US24/32910, filed on Jun. 7, 2024.
(60) Provisional application No. 63/472,050, filed on Jun. 9, 2023.

(51) Int. Cl. A61K 31/4995; A61P 7/10; A61P 17/00

(52) U.S. Cl. CPC A61K 31/4995; A61P 7/10; A61P 17/00

(57) ABSTRACT

Described are methods of treating lipedema in a patient in need thereof comprising administering a selective LTA4H inhibitor in a therapeutically effective amount. Also described are methods of treating a lymphatic dysfunction-associated abnormality of subcutaneous tissue or lymphatic disease of the subcutaneous tissue in a patient in need thereof comprising administering to said patient a therapeutically effective amount of a selective LTA4H inhibitor. In addition, methods of preventing or lowering the risk of developing lymphedema in a patient in need thereof are described.

### RELATED APPLICATIONS

This application is a continuation of International Application No. PCT/US24/32910, which designated the United States and was filed on Jun. 7, 2024, published in English, which claims the benefit of U.S. Provisional Application No. 63/472,050 filed Jun. 9, 2023. The entire contents of the above-referenced applications are incorporated by reference herein.

### BACKGROUND OF THE INVENTION

Lipedema is a chronic condition estimated to affect about 10% of women (Kruppa et al. (2020)). The condition is characterized by deposition of body fat in the extremities that is bilateral and that persists despite diet and exercise (Kruppa et al.; Escobedo et al. (2017)). Lipedema is also associated with painful subcutaneous adipose tissue (SAT). The SAT is morphologically abnormal, including the presence of hypertrophic adipocytes (Poojari et al. (2022)). Ultrasound of the SAT of lipedema patients shows abnormal ultrasonographic fascial planes (e.g., a loss of distinction or blurring of fascial interfaces in the fat tissue) and changes in echogenicity as compared to that in normal patients (Ibarra et al. (2018); Iker et al. (2019)). The blurring of the fascia is believed to be due to edema in the tissue (Ibarra et al.). Bilancini et al. demonstrated that lipedema is consistently associated with functional alterations of the lymphatic vasculature. Using dynamic imaging, they showed that patients suffering from lipedema have an abnormal lymphoscintigraphic pattern, with a slowing of the lymphatic flow, similar to the alterations found in lymphedema patients. Thus, lipedema is an adipose disorder that is associated with functional alterations of the lymphatic system (Escobedo et al. (2017); Poojari et al. (2022); Kruppa et al.). Lymphatic dysregulation has been described as both a consequence and a causal factor of lipedema and adipose tissue expansion (Poojari et al. (2022)).

Lipedema is frequently misdiagnosed as obesity as well as lymphedema. Lymphedema, which affects as many as 200 million patients worldwide, is also associated with dysfunction of the lymphatic system and abnormal SAT. Lymphedema is caused by relative lymphatic vascular insufficiency and is characterized by increasing limb girth, fibrosis, inflammation, and marked cutaneous pathology that increases the risk of recurrent skin infections. Like lipedema, lymphedema is associated with the abnormal distribution of subcutaneous fat in the extremities (Escobedo et al. (2017)). Ultrasound of the affected tissue in lymphedema patients shows changes in dermal and SAT echogenicity as compared to that in normal patients, blurring of the interface between the subcutaneous fat and the skin, as well a loss of distinctness of the fascia lines (Iker et al. (2019); Suehiro et al. (2013)).

There remains a need in the art for pharmacologic treatment of lipedema as well as the abnormal adipose tissue of lipedema and lymphedema patients.

### SUMMARY OF THE INVENTION

The present invention is at least partially based on the discovery that treatment with acebilustat resulted in early, profound ultrasonographic skin changes in the affected limb of a secondary lymphedema patient. Specifically, as shown in the Examples and figures, acebilustat reduced dermal thickness, subcutaneous thickness, limb volume, water content, and subcutaneous echogenicity of the affected limb as seen by ultrasound. Reduced dermal thickness and subcutaneous echogenicity suggest that acebilustat may target inflammation and excess water content of affected tissues. These findings have relevance to tissue changes observed in lipedema as well as lymphedema.

The present invention includes methods of treating lipedema in a patient in need thereof comprising administering acebilustat or another selective LTA4H inhibitor in a therapeutically effective amount. A patient suffering from lipedema can, for example, be a patient suffering from combined pathologies, for example, Stage IV lipedema, also known as lipolymphedema). In certain specific aspects, the method comprises administering acebilustat. In yet additional aspects, pain, bruising, and/or capillary fragility is reduced after treatment with acebilustat; for example, pain, bruising and/or capillary fragility is reduced after treatment with acebilustat for six months or less, or three months or less.

Also described herein are methods of treating a lymphatic dysfunction-associated abnormality of subcutaneous tissue or lymphatic disease of the subcutaneous tissue in a patient in need thereof comprising administering to said patient a therapeutically effective amount of acebilustat or another selective LTA4H inhibitor. In certain aspects, the patient is suffering from lipedema. In additional aspects, the patient is suffering from lymphedema. A patient suffering from lymphedema can be a patient suffering from combined pathologies, for example, Stage IV lipedema, also known as lipolymphedema. In yet additional aspects, the patient is suffering from lower limb lymphedema. In further aspects, the patient is suffering from upper limb lymphedema. In certain additional aspects, the invention is a method of treating a lymphatic dysfunction-associated abnormality of subcutaneous tissue or lymphatic disease of the subcutaneous tissue in a patient is suffering from lymphedema, wherein the subdermal thickness of the affected area (the area affected by the lymphatic dysfunction-associated abnormality of subcutaneous tissue or the lymphatic disease of the subcutaneous tissue) is reduced by at least about 10% after administration of acebilustat for three months or less as compared to baseline.

In yet further aspects, the invention is a method of reducing the subdermal thickness of a lymphedematous area in a patient suffering from lymphedema, the method comprising administering to said patient a therapeutically effective amount of acebilustat, wherein the subdermal thickness is reduced by at least about 10% after the administration of acebilustat for six months or less, as compared to baseline. In yet additional aspects, the subdermal thickness is reduced by at least about 10% after the administration of acebilustat for three months or less. In yet further embodiments, the subdermal thickness is reduced by at least about 15% or at least about 20% after the administration of acebilustat for six months or less, as compared to baseline. In yet further aspects, the subdermal thickness is reduced by at least about 15% or at least about 20% after the administration of acebilustat for three months or less, as compared to baseline.

In certain aspects, the invention is a method of treating lymphatic dysfunction-associated abnormality of subcutaneous tissue or lymphatic disease of the subcutaneous tissue, or lymphedema in a patient in need thereof, the method comprising administering to the patient a therapeutically effective amount of acebilustat or another selective LTA4H inhibitor and wherein the patient is further treated with a surgical intervention including, for example, lymphovenous anastomosis (LVA), vascularized lymph node transfer (VLNT), and implantation of an artificial lymph node. In certain aspects, the lymphedema is an upper limb lymphedema. In additional aspects, the lymphedema is a lower limb lymphedema. In certain aspects, acebilustat is administered.

The invention additionally encompasses a method of preventing or lowering the risk of developing lymphedema in a patient in need thereof comprising administering to said patient an effective amount of acebilustat or another selective LTA4H inhibitor. In certain aspects, acebilustat is administered. In some examples, the patient in need of prevention or lowering the risk of developing lymphedema can be a cancer patient and the patient that is undergoing or has undergone lymph node dissection or radiation therapy, for example, as part of cancer treatment. In certain embodiments, the acebilustat is administered in combination with another prophylactic intervention, for example, LVA or implantation of an artificial lymph node.

In yet additional embodiments, the invention includes a method of promoting lymphangiogenesis and/or lymphatic repair in a patient in need thereof comprising administering to said patient an effective amount of acebilustat or another selective LTA4H inhibitor. In certain aspects, acebilustat is administered. For example, the patient in need of promoting lymphangiogenesis and/or lymphatic repair is a patient at risk of developing lymphedema, for example, a cancer patient or patient undergoing or that has undergone lymph node dissection or radiation therapy (for example, as part of cancer therapy). In yet additional aspects, the patient in need of promoting lymphangiogenesis and/or lymphatic repair is a patient implanted with an artificial lymph node; in certain aspects, treatment with acebilustat increases lymphatic growth and repair to and from the artificial lymph node. In yet further aspects, the invention is a method of promoting lymphangiogenesis and/or lymphatic repair to an artificial lymph node in a patient implanted with the artificial lymph node, the method comprising administering to said patient an effective amount of acebilustat.

The invention also includes a method of treating lymphedema in a patient in need thereof, wherein the subcutaneous echogenicity of the affected (lymphedematous) area is increased as compared to normal (in other words, the lymphedema is characterized by a subcutaneous echogenicity of the affected area that is increased as compared to normal), the method comprising administering to the patient an effective amount of acebilustat or another selective LTA4H inhibitor, and wherein echogenicity is determined by ultrasound. In certain aspects, the method further comprises determining dermal structural attributes by ultrasound. In certain aspects, acebilustat is administered.

The invention additionally includes a method of treating lymphedema in a patient in need thereof, wherein the lymphedema is characterized by an affected area or affected tissue that has a dermal echogenicity to subcutaneous echogenicity ratio that is less than a control ratio, the method comprising administering to the patient an effective amount of acebilustat or another selective LTA4H inhibitor, and wherein echogenicity determined by ultrasound. In certain aspects, the method further comprises determining dermal structural attributes by ultrasound. In certain aspects, acebilustat is administered.

The invention also includes a method of identifying a patient responsive to treatment for lymphedema, the method comprising:
i. determining dermal echogenicity and subcutaneous echogenicity of the affected area or affected tissue by ultrasound;
ii. calculating the ratio of the dermal echogenicity to the subcutaneous echogenicity of the affected area or the affected tissue;
iii. identifying the patient as responsive to treatment if the ratio of the affected area is less than a control ratio; and
iv. administering acebilustat or another selective LTA4H inhibitor to the patient identified as responsive to the treatment.
   In certain aspects, acebilustat is administered.

Also disclosed herein is a method of identifying a lymphedema patient responsive to treatment with acebilustat, the method comprising:
i. determining dermal echogenicity and subcutaneous echogenicity of the affected area or affected tissue by ultrasound;
ii. calculating the ratio of the dermal echogenicity to the subcutaneous echogenicity of the affected area or the affected tissue;
iii. identifying the patient as responsive to treatment if the ratio of the affected area is less than a control ratio; and
iv. administering acebilustat to the patient identified as responsive to the treatment.

The invention also encompasses a method of treating lymphedema in a patient in need thereof, the method comprising:
i. determining the dermal echogenicity and the subcutaneous echogenicity of the affected (lymphedematous) area or affected (lymphedematous) tissue by ultrasound at baseline and calculating the ratio of the dermal echogenicity to the subcutaneous echogenicity at baseline;
ii. administering acebilustat or another selective LTA4H inhibitor to said patient at an initial daily dose, for example, for at least about three months, at least about four months, at least about five months, at least about six months, at least about nine months, or for at least about one year;
iii. determining dermal echogenicity and subcutaneous echogenicity of the affected area or the affected tissue by ultrasound after treatment and calculating the ratio of the dermal echogenicity to the subcutaneous echogenicity after treatment; and
iv. continuing administration of acebilustat or the other selective LTA4H inhibitor at the initial daily dose if the ratio after treatment is greater than the ratio at baseline, or increasing the daily dose of acebilustat to an increased daily dose if the ratio after treatment is not greater than the ratio at baseline.
   In certain aspects, acebilustat is administered.

In specific embodiments, the methods comprise administering to the patient an effective amount of acebilustat. For example, the acebilustat can be administered at least once a day, for example orally. In certain embodiments, the methods comprise orally administering to patients acebilustat at a total daily dose of about 200 mg or less, about 150 mg or less, about 100 mg or less, about 50 mg or less, from about 50 mg to about 100 mg, about 100 mg, or about 50 mg.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A, 1B and 1C are graphs showing skin thickness (mm) of the affected (lymphedematous) limb over time; measured at baseline, 3 months and at 6 months after acebilustat treatment for the 10 or 11 patients as shown. Skin thickness was measured by caliper. As early as 3 months after acebilustat treatment, a significant decrease in skin thickness was observed (p<0.0001).

FIGS. 2A, 2B and 2C are graphs presenting the same data above as percent change in skin thickness (as compared to baseline) over time; measured at baseline, 3 months and at 6 months after acebilustat treatment for 10 or 11 patients as shown. At 6 months, a 43% reduction in skin thickness was observed (mean for 10 patients; p<0.0001).

FIGS. 3A, 3B and 3C are graphs showing limb volume (% change) over time; measured at baseline, 3 months and at 6 months after acebilustat treatment for 6-11 patients as shown. At 6 months, a 6.7% reduction in limb volume was observed (mean for 6 patients; p<0.03).

FIGS. 4A, 4B and 4C are graphs showing bioimpedance (mOhm) of the affected limb over time (at baseline and at 3 months and 6 months after acebilustat treatment). Bioimpedance is a measure of water content, and a higher bioimpedance corresponds to a lower water content.

FIGS. 5A, 5B and 5C are graphs presenting the same data as change in bioimpedance (as compared to baseline) over time. At 6 months (FIG. 5C), a significantly higher bioimpedance (corresponding to lower water content) was observed (p<0.01).

FIGS. 6A and 6B are dermal ultrasound images of a patient with right leg lymphedema prior to treatment (FIG. 6A; left), and after 3 months of treatment with acebilustat (FIG. 6B; right). Subdermal thickness was reduced by over 20.2%, dermal thickness was reduced by 3.7%, and total thickness was reduced by 17.6% at 6 months (6 months of treatment with acebilustat).

FIG. 7 shows ultrasound images of a different patient than that of FIGS. 6A and 6B and shows subcutaneous thickness pre-treatment (left) and after 6 months of treatment with acebilustat (right). A 15.2% reduction in subcutaneous thickness was observed after 6 months of treatment.

FIG. 8 shows graphs showing LymVAS Total Status Score, a tool to assess patient quality of life, at 3 months and 6 months after acebilustat treatment. A significant improvement in LymVAS quality of life Status Score was observed as early as 3 months after acebilustat treatment.

FIG. 9 shows graphs showing LymVAS Total Change score, which is a tool to assess change in patient quality of life, at 3 and 6 months after acebilustat treatment. A significant improvement in LymVAS total Change Score was observed as early as 3 months after acebilustat treatment.

### DETAILED DESCRIPTION OF THE INVENTION

A description of preferred embodiments of the invention follows.

As used herein, the words “a” and “an” are meant to include one or more unless otherwise specified. For example, the term “an additional therapeutic agent” encompasses both a single additional therapeutic agent and a combination of two or more additional therapeutic agents. In another example, “an upper limb” is meant to include one or both arms.

It is to be understood that when the range of the dose or amount of a drug or active ingredient (e.g., acebilustat) is described as “between” a low end of the range and “between” a high end of the range, the range is meant to include both, the low end and the high end as well as doses in between the low and high ends. For example, for “a dose between about 50 mg and about 100 mg,” it is to be understood that the range includes the low end of the range, about 50 mg, and the high end of the range, about 100 mg, as well as the doses in between, for example, about 75 mg. In addition, “a dose of about 50 mg or less” is intended to include the about 50 mg dose as well as doses less than about 50 mg.

The term “about” as used herein, in reference to a numerical value or range, allows for a degree of variability in the value or range, for example, within 10%, within 5%, or within 4%, or within 2% of the value or range, or within 1% of the value or range.

The term “affected area” or in reference to a lipedema patient refers that part of the patient's anatomy that is lipedematous. For example, in Type III lipedema, the affected area can be the thighs or a portion thereof. In a lipedema patient, the “affected tissue” refers to the tissue of the part of the patient's anatomy that is affected by the disease (that is lipedematous). The term “affected tissue” and the like can be used interchangeably with the terms, “lipedematous tissue” and “lipedema tissue” and the like. For example, in Type III lipedema, the affected tissue can be the subcutaneous tissue of the affected thigh area.

The term “affected area” in reference to a lymphedema patient refers to that part of the patient's anatomy that is lymphedematous. The affected area can be one or both arms, and/or can be one or both legs. In a lymphedema patient, the terms “affected tissue” and the like refers to the tissue of the part of the patient's anatomy that is affected by the disease (that is lymphedematous). The term “affected tissue” can be used interchangeably with the terms, “lymphedematous tissue” and “lymphedema tissue” and the like. The term “tissue” includes the epifascial layers of the skin.

The term “affected tissue” in reference to a patient with lymphatic disease of the subcutaneous tissue or lymphatic-dysfunction associated abnormality of the subcutaneous tissue is the subcutaneous tissue of the patient that exhibits the lymphatic disease and/or the lymphatic-dysfunction associated abnormality.

Echogenicity of tissue is the ability of the tissue to reflect or transmit ultrasound waves in the context of the surrounding tissues (Ihnatsenka et al. (2020)). A structure or tissue (or portion thereof) can be characterized as hyperechoic (white on the ultrasound image), hypoechoic (gray on the ultrasound image) and anechoic (black on the ultrasound image). For example, fat is almost anechoic, while fascia and other connective tissue strands usually appear as hyperechoic lines. In addition, when there is an interface between two different structures (e.g., the dermis and the epidermis), a visible difference in contrast will usually be apparent. As described in more detail herein, lipedema and lymphedema are associated with certain specific ultrasonographic changes as compared to normal unaffected tissues, including a change in echogenicity. Echogenicity can, for example, be measured at a specific region of interest (ROI) within the tissue.

The “dermal echogenicity to subcutaneous echogenicity ratio” is the ratio of the echogenicity of the dermis (or a region of interest in the dermis) to the echogenicity of the subcutaneous tissue (or a region of interest in the subcutaneous tissue). In certain aspects, the dermal to subcutaneous echogenicity ratio is the echogenicity of the dermis (or a region of interest in the dermis) to the echogenicity of the subcutaneous adipose tissue (or a region of interest in the subcutaneous adipose tissue). The baseline dermal echogenicity to subcutaneous echogenicity ratio is determined in the same subject or a similar subject population before treatment is initiated. When the baseline dermal echogenicity to subcutaneous echogenicity ratio is determined using a similar subject population to the patient, the subject population can be made up of individuals suffering from the same disease as the patient, and optionally, the same stage or type. For example, for lipedema, this can be the mean dermal echogenicity to subcutaneous echogenicity ratio of a population of patients suffering from lipedema similar in type and/or stage to the patient. For lymphedema, this can be the mean dermal echogenicity to subcutaneous echogenicity ratio of a population of patients suffering from lymphedema similar in type to that of the patient. The subject population can optionally be demographically similar, for example, similar in age or sex. The dermal echogenicity and the subcutaneous echogenicity used to calculate the ratio can be measured/determined using the same ultrasound device (e.g., at baseline and/or after treatment).

The present invention is directed to the use of acebilustat, for example, in patients diagnosed with lipedema as well as lymphatic disease of the subcutaneous tissue and/or lymphatic dysfunction-associated abnormality of the subcutaneous tissue. Such lymphatic disease of the subcutaneous tissue is observed in most patients with lymphedema as well as universally in lipedema. It is believed that acebilustat inhibits the enzymatic production of LTB4 in such patients, and, as a consequence of this amelioration in molecular signaling, can reverse the abnormal fat distribution, loss of function, and/or impaired quality-of-life experiences in such patients.

The invention encompasses methods of treating a lymphatic dysfunction-associated abnormality of subcutaneous tissue or lymphatic disease of the subcutaneous tissue in a patient in need thereof, comprising administering to said patient a therapeutically effective amount of acebilustat. In certain aspects, the subcutaneous tissue comprises subcutaneous adipose tissue (SAT). In additional aspects, the subcutaneous tissue is SAT. In additional aspects, the subcutaneous tissue comprises or consists of fibrotic tissue. The lymphatic dysfunction-associated abnormality can, for example, be an accumulation or deposition of SAT, inflammation of the SAT, adipose hypertrophy and/or hyperplasia, and/or a disruption of the fascial bands of the SAT, or a combination thereof. In certain aspects, the patient with a lymphatic dysfunction-associated abnormality of subcutaneous tissue or lymphatic disease of the subcutaneous tissue is suffering from lymphedema. In additional aspects, the patient with a lymphatic dysfunction-associated abnormality of subcutaneous tissue or lymphatic disease of the subcutaneous tissue is suffering from lipedema.

The invention also encompasses a method of preventing or reducing the risk of a lymphatic dysfunction-associated abnormality of subcutaneous tissue or lymphatic disease of the subcutaneous tissue in a patient at risk thereof, comprising administering to said patient a therapeutically effective amount of acebilustat. In certain aspects, the subcutaneous tissue comprises subcutaneous adipose tissue (SAT). In additional aspects, the subcutaneous tissue is SAT. In additional aspects, the subcutaneous tissue comprises or consists of fibrotic tissue. The lymphatic dysfunction-associated abnormality can, for example, be an accumulation or deposition of SAT, inflammation of the SAT, adipose hypertrophy and/or hyperplasia, and/or a disruption of the fascial bands of the SAT, or a combination thereof. In certain aspects, the patient with a lymphatic dysfunction-associated abnormality of subcutaneous tissue or lymphatic disease of the subcutaneous tissue is suffering from lymphedema. In additional aspects, the patient with a lymphatic dysfunction-associated abnormality of subcutaneous tissue or lymphatic disease of the subcutaneous tissue is suffering from lipedema.

The methods described herein can improve one or more symptoms, characteristics, endpoints, or ultrasonographic characteristics of the disease being treated (e.g., lipedema, lymphedema, lymphatic dysfunction-associated abnormality of subcutaneous tissue, or lymphatic disease of the subcutaneous tissue). The improvement or change can be observed, for example, about 1 month, about 2 months, about 3 months, about 4 months, about 5 months, about 6 months, about 9 months or about 1 year or more after treatment (e.g., administration of acebilustat or other LTA4H inhibitor) is initiated. The improvement or change can be observed, for example, within about 1 month, within about 2 months, within about 3 months, within about 4 months, within about 5 months, within about 6 months, within about 9 months, about 1 year, or within 2 years after treatment (e.g., administration of acebilustat or other LTA4H inhibitor) is initiated.

The methods of preventing or reducing the risk of a lymphatic dysfunction-associated abnormality of the subcutaneous tissue or lymphatic disease of the subcutaneous tissue in a patient at risk thereof can protect the patient from developing lymphatic dysfunction-associated abnormality of the SAT or lymphatic disease of the SAT, or symptoms of either of thereof, prevent the lymphatic dysfunction-associated abnormality of the SAT or lymphatic disease of the SAT, or symptoms of either of thereof from developing, or reduce the risk that the lymphatic dysfunction-associated abnormality of the SAT or lymphatic disease of the SAT, or symptoms of either of thereof will develop, for example, as compared to the patient's risk in the absence of the acebilustat administration. The methods encompass preventing or reducing the risk of lipedema or lymphedema in a patient in need thereof. The methods of preventing or reducing the risk of lipedema in a patient at risk thereof can protect the patient from developing lipedema, symptoms thereof, prevent lipedema or symptoms thereof from developing, or reduce the risk that the lipedema will develop, for example, as compared to the patient's risk in the absence of the acebilustat administration. The methods of preventing or reducing the risk of lymphedema in a patient at risk thereof can protect the patient from developing lymphedema, symptoms thereof, prevent lymphedema or symptoms thereof from developing, or reduce the risk that the lymphedema will develop, for example, as compared to the patient's risk in the absence of the acebilustat administration.

When a symptom or endpoint is described as having improved after administration of an active agent “for X months or less,” this is intended to encompass the improvement of the symptom or endpoint determined at a timepoint that is about X months after initiation of the active agent treatment as well as the improvement determined at a timepoint that is less than X months after initiation of acebilustat treatment. For example, a reduction in subcutaneous thickness “after administration of acebilustat for 6 months or less” and the like encompasses the reduction in subcutaneous thickness measured at a timepoint that is about 6 months after initiation of the acebilustat treatment as well as the reduction measured at a timepoint that is less than 6 months after initiation of the acebilustat treatment.

Lipedema is classified in Stages (stages I to IV) and types (I to V). Stage I lipedema is characterized by normal skin texture over an enlarged subdermis (Vyas et al., 2023). In Stage 2, the skin has an uneven surface over hypodermal masses (Id.). Stage 3 is characterized by more painful and increased lipedema tissue and a loss of skin elasticity (Poojari et al.). In Stage 4, patients have concomitant lymphedema, termed lipolymphedema. Types of lipedema are classified based on the affected anatomies. Type I includes fat distribution from the umbilicus to the hips, including the pelvis and buttocks. Type 11 includes fat distribution from around the pelvis to the knees. Type III includes fat distribution from around the pelvis to ankles with a prominent cuff at the ankle. Type IV includes fat distribution from the shoulders to the wrists. Type V is rare and includes fat distribution from or below knees extends through ankles. Patients can exhibit combinations of lipedema types. For example, patients with Types II and IV and patients with Types II and IV are common. The invention encompasses methods of treating Stage 1, Stage 2, Stage 3 and/or Stage 4 lipedema. The invention also encompasses methods of treating Type I, Type 1I, Type 111, Type IV and/or Type V lipedema, as well as combinations thereof.

The treatment described herein can improve one or more symptoms, characteristics, endpoints, or ultrasonographic characteristics of the disease. The improvement or change can be observed, for example, about 1 month, about 2 months, about 3 months, about 4 months, about 5 months, about 6 months, about 9 months or about 1 year, or more after treatment (e.g., administration of acebilustat) is initiated. Such improvement can be compared to that before treatment is initiated (referred to herein as “baseline”). For example, the treatment can decrease the severity of the disease and/or or improve skin histology and/or function and/or elasticity as compared to that at baseline. In another example, treatment with acebilustat can reduce inflammation and/or reduce pain and/or reduce capillary fragility and/or reduce bruising. In an additional example, treatment with acebilustat can increase lymphatic flow and/or improve vascular function, and/or increase lymphatic drainage, and/or decrease edema. In a further aspect, the treatment results in an improvement in a lymphatic symptom as compared to that at baseline, for example, as demonstrated by lymphoscintigraphy and indocyanine green (ICG) lymphography. Lymphoscintigraphy findings in lipedema include convoluted lymphatic vessels in the legs that slow transit of radionuclide (Herbst et al. (2021)). In yet further aspects, the treatment results in a reduction in the amount of fat or adipose in the affected area and/or structural changes in the adipose tissue of the affected area. Additionally, treatment as described herein can decrease the number or size of lipedema nodules.

As described above, treatment with acebilustat can reduce pain and/or bruising in patients with lipedema. Symptoms of lipedema include pain (e.g, pain upon palpation), easy bruising, and disfigurement. Lipedema pain has also been described as tenderness. The term “lipedema pain” is inclusive of tenderness and discomfort, for example, in one or both arms and/or pain/tenderness/discomfort in one or both legs. In certain embodiments, treatment with acebilustat as described herein reduces lipedema pain. For example, lipedema pain is reduced in the affected area after administration of acebilustat for six months or less, or three months or less. Lipedema pain can be measured, for example, by subjective pain scores as described, for example, in Smoot et al. (2015), J Cancer Surviv. 9: 287-304; the contents of which are expressly incorporated by reference herein. In yet further aspects, lipedema-associated bruising and/or hematoma formation is reduced in the affected area after administration of acebilustat for six months or less, or three months or less.

Lipedema is also associated with increased capillary fragility. In certain embodiments, treatment with acebilustat improves or reduces capillary fragility, for example, as measured by the vacuum suction method (VSM) using Parrot's angiosterrometer as described, for example, Szolnoky et al. (2017), Lymphology 50(4): 203-209. Capillary fragility is the tendency to capillary wall rupture. An increased infiltration of macrophages is observed in lipedema tissue, and the infiltrating macrophages are predominantly M2-polarized macrophages (Wolf et al. (2022)). In certain embodiments, the treatment reduces the number of macrophages and/or reduces the number of M2-polarized macrophages. In additional aspects, the treatment can change the macrophage composition. In additional aspects, the treatment reduces the number of fibroblasts in the lipedema tissue.

As described above, lipedema in some patients is associated with decreased visualization of the fascial bands/planes of the affected subcutaneous tissue (e.g., the subcutaneous adipose tissue) under ultrasound, and/or changes in echogenicity of the dermal and/or subcutaneous tissues. In certain aspects, treatment with acebilustat results in ultrasonographic changes in the affected (lipedematous) area or tissue as compared to that at baseline. As used herein “baseline” is a timepoint prior to the initiation of the treatment (e.g., acebilustat or other LTA4H inhibitor). Baseline echogenicity is determined in the same subject or a similar subject population (e.g., a population of patients suffering from lipedema similar in type and/or stage) before treatment is initiated. Treatment with acebilustat can result in a decrease in the subcutaneous echogenicity (for example, the echogenicity of the subcutaneous adipose tissue or a region of interest in the SAT) of the affected (lipedematous) area or tissue as compared to baseline. For example, there may be heterogeneity in the subcutaneous lipedema tissue with regions of increased echogenicity at baseline, and the echogenicity of one or more of these regions of interest is decreased after treatment with acebilustat. In additional aspects, the treatment described herein results in an improvement and/or visualization by ultrasound of the fascia bands of the subcutaneous adipose tissue of the affected area. In yet further aspects, the dermal echogenicity to subcutaneous echogenicity ratio of the lipedematous tissue is increased after treatment with acebilustat or another selective LTA4H inhibitor as compared to that at baseline.

While the genetic components of the disease are not yet fully elucidated, lipedema is likely an inherited, autosomal dominant disease. The patient to be treated with acebilustat can, for example, be a patient that expresses a biomarker indicative of lipedema, for example, an increased level of PF4 has been reported in lipedema and lymphedema patients (as described, for example, in US20220107329, the contents of which are expressly incorporated by reference herein). In additional aspects, the patient to be treated can be a carrier for a gene associated with lipedema. Such a gene includes, for example, Aldo-Keto Reductase 1C1 (AKR1C1) where a mutation (e.g., Leu213Gln) is associated with nonsyndromic primary lipedema (Michelini et al. (2020)). Additional genetic factors have been described, for example, in Paolacci et al. (2019), Genetics of lipedema: new perspectives on genetic research and molecular diagnoses, Eur Rev Med Pharmacol Sci 23(13):5581-5594; the contents of which are expressly incorporated by reference herein. The invention also includes a method of preventing, decreasing the risk of developing lipedema, or inhibiting progression of lipedema in an individual to be treated that has, or that has been, or is being treated for lipedema, or a patient at risk of developing lipedema, for example a patient that expresses a genetic marker associated with lipedema.

In certain aspects, the patient is treated with surgery, for example, lipedema reduction surgery in addition to administration of the acebilustat or other selective LTA4H inhibitor before or after the surgery. In yet further aspects, the patient is additionally treated with compression therapy, physiotherapy, exercise therapy, functional lymphological rehabilitation and/or manual lymphatic drainage. In further aspects, the patient is treated with an additional active agent selected from the group consisting of sympathomimetics (e.g., amphetamine or phentermine), metformin, resveratrol, diosmin and selenium, or a combination thereof, in addition to the administration of acebilustat. The acebilustat or other selective LTA4H inhibitor can be administered simultaneously with, prior to, or after administration of one or more additional active agents. Such combination therapy includes administration of a single pharmaceutical dosage formulation which contains the acebilustat or other selective LTA4H inhibitor and the one or more additional active agents, as well as administration of the acebilustat and each active agent in its own separate pharmaceutical dosage formulation.

The invention also includes a method of treating lipedema in a patient in need thereof, wherein the subcutaneous echogenicity of the affected area or tissue (or a region of interest) at baseline is increased as compared to normal, the method comprising administering to the patient an effective amount of acebilustat. “Normal” echogenicity in this context means the echogenicity of the same part of the anatomy or a corresponding region of interest in an individual (or individuals) not suffering from lipedema, or in other words, a normal individual or a population of normal individuals.

The invention additionally includes a method of treating lipedema in a patient in need thereof, wherein the affected area (or a portion thereof) has a dermal echogenicity to subcutaneous echogenicity ratio at baseline that is less than a control ratio, the method comprising administering to the patient an effective amount of acebilustat, and wherein echogenicity is determined by ultrasound. The “control ratio” is the mean dermal echogenicity to subcutaneous echogenicity ratio of a population of normal individuals. In certain aspects, the treatment is initiated or acebilustat is administered to said patient if the ratio (for example, at baseline) is at least 5%, 10%, 15% or 20% lower than the control ratio.

Also encompassed is a method of treating lipedema in a patient in need thereof, the method comprising:
determining dermal echogenicity and subcutaneous echogenicity of the affected (lipedematous) area or affected tissue by ultrasound at baseline and calculating the ratio of dermal echogenicity to subcutaneous echogenicity at baseline;
administering acebilustat to said patient at an initial daily dose; and
determining the dermal echogenicity and the subcutaneous echogenicity of the affected area or the affected tissue by ultrasound after treatment and calculating the ratio of dermal echogenicity to subcutaneous echogenicity after treatment;
continuing administration of acebilustat at the initial daily dose if the ratio after treatment is greater than the ratio at baseline, or increasing the daily dose of acebilustat to an increased daily dose if the ratio after treatment is not greater than the ratio at baseline. The initial daily dose is the daily dose at the time of treatment initiation and/or daily dose at the time that the dermal echogenicity and subcutaneous echogenicity are measured. The increased daily dose is a daily dose that is greater than the initial daily dose.

The dermal echogenicity and subcutaneous echogenicity can be determined after treatment, for example, at least about one month, at least about two months, at least about three months, at least about four months, at least about five months, at least about six months, at least about nine months, at least about one year, at least about 18 months or at least about 2 years after initiating administration of acebilustat. If the ratio is not increased (at the time point at which it is measured, e.g., 6 months after initiating the treatment), then the dose regimen can be modified, for example, the total daily dose can be increased.

Acebilustat or the other selective LTA4H inhibitor can be administered to a patient in addition to the existing treatment regime, or in addition to the standard of care. The standard of care for lipedema includes, but is not limited to, exercise (such as postural core exercises and muscle strengthening exercises), gait training, neuromuscular re-education, abdominal breathing to increase lymphatic flow, manual therapy, compression garments, pneumatic compression device, soft tissue mobilization (e.g., to reduce pain and inflammation), manual lymphatic drainage, and surgery. Treatment of lipedema can also include administration of an agent selected from the group consisting of sympathomimetics, metformin, resveratrol, diosmin and selenium, or a combination thereof; therefore, the acebilustat or other LTA4H inhibitor can be administered to a patient in addition to an agent selected from the group consisting of sympathomimetics, metformin, resveratrol, diosmin and selenium, or a combination thereof. For example, the sympathomimetic is selected from the group consisting of amphetamine and phentermine. In certain additional aspects, the acebilustat or other LTA4H inhibitor is administered to a patient being treated with glucagon-like peptide (GLP-1) agonist including, but not limited to, dulaglutide, exenatide, semaglutide, liraglutide, terzepatide, and lixisenatide.

In yet another aspect, the acebilustat or other selective LTA4H inhibitor is administered in combination with one or more additional active agents useful in preventing or treating lipedema. In some of these embodiments, the acebilustat or other selective LTA4H inhibitor is administered with a different LTB₄ inhibitor. For example, the different LTB4 inhibitor can be of the BLT1/BLT2 antagonist classes of LTB₄ inhibitors. In additional examples, the acebilustat or other selective LTA4H inhibitor is administered in combination with sympathomimetics (e.g, amphetamine or phentermine), metformin, resveratrol, diosmin and selenium.

For lipedema, treatment, for example, can commence at any stage of the condition and continue until or past where the treatment stabilizes or reverses patient condition to a non-symptomatic state. Treatment with the methods of the invention can, for example, commence following onset of any clinically evident lipedema, such as stage 1. Treatment with the methods of the invention can also, for example, commence following onset of stage 2 lipedema. Treatment with the methods of the invention can commence following onset of stage 3 lipedema. Treatment can also commence before the onset of lipedema but in a patient at risk of developing lipedema.

Those of skill in the art will appreciate that the methods of the invention are applicable to the treatment and prevention of lipedema including its signs and symptoms such as those associated with the following clinical indicia of lipedema. A number of clinical indicia can be used to diagnose lipedema and to monitor the effectiveness of therapy, including treatment with the compositions and methods of the present invention. The invention provides methods of determining efficacy of a lipedema treatment in a subject in need thereof by (a) measuring an endpoint of a clinical indication in a patient, where the endpoint is measured after treatment has started, (b) comparing the endpoint of the clinical indication to a baseline or reference, where the baseline or reference is measured in the same subject or a similar subject population before treatment is begun, and (c) determining the efficacy of the lipedema treatment based on the comparison step. Analysis of clinical indicia may include ultrasonographic changes; reduction of subcutaneous adipose tissue; structural change in subcutaneous adipose tissue, reduction in edema or swelling, reduction in pain, reduction in bruising, reduction in capillary fragility, and/or change of lymphatic architecture and function.

In some embodiments of the invention, e.g., treatment of patients with established disease, improvements in the architecture of the adipose tissue of the affected area provide a convenient method for assessing treatment success. In some embodiments, a treatment provided herein is efficacious if, after a period of time from the onset of treatment (e.g., about 2 weeks, about 1 month, about 2 months, about 3 months, about 4 months, about 5 months, about 6 months or longer), there is a decrease in fat deposition or change in the architecture of the adipose tissue (as measured by ultrasound) of at least one affected region as compared to that of the at least one affected region prior to the onset of treatment.

The invention also encompasses methods of treating lymphatic-dysfunction associated abnormality of the subcutaneous tissue or lymphatic disease of the subcutaneous tissue in a lymphedema patient. Methods for the treatment of lymphedema using acebilustat have been described, for example, in WO2023/107608 (PCT Application No. PCT/US22/52230; entitled “Methods of Treating Lymphedema”); the contents of which are expressly incorporated by reference herein. In addition, methods for the treatment of lymphedema using the LTA4H inhibitor, ubenimex, have been described for example in U.S. Pat. No. 10,500,178; the contents of which are expressly incorporated by reference herein. Lymphedema, as used herein, is edema of a region or regions of the body due to genetically determined lymphatic dysfunction (primary lymphedema) or to obstruction, disruption or dysfunction (secondary or acquired lymphedema) of lymphatic vessels. Symptoms and signs can comprise varying degrees of brawny, fibrous, non-pitting edema in one or more regions of the body. In certain aspects, the patient is suffering from primary lymphedema. In additional aspects, the patient is suffering from secondary lymphedema. In yet further aspects, the lymphedema is cryptogenic lymphedema.

Primary lymphedemas are constitutional and relatively less common than the secondary forms. They vary in phenotype and patient age at presentation. The methods of the invention are applicable to these primary forms of lymphedema, although it will be understood by one of skill in the art that treatment may be more efficacious in some forms than others due to the differing disease etiologies. Primary forms of lymphedema include, without limitation Milroy's disease, Meige disease, (lymphedema praecox), lymphedema distichiasis, lymphedema tarda, etc., as well as other genetic syndromes having prominent lymphedema, such as Turner's syndrome and Hennekam syndrome. For example, congenital lymphedema appears at birth or within months thereafter, and may be due to lymphatic aplasia or hypoplasia. Milroy's disease is an autosomal dominant familial form of congenital lymphedema attributed to FLT4 gene mutations and associated with edema and, sometimes, diarrhea and/or hypoproteinemia due to a protein-losing enteropathy caused by intestinal lymphangiectasia. Lymphedema distichiasis is an autosomal dominant familial form of lymphedema praecox attributed to mutations in a transcription factor gene (FOXC2) and associated with extra eyelashes (distichiasis), and edema of legs, arms, and sometimes the face. Lymphedema tarda appears after age 35. Both familial and sporadic forms exist; the genetic basis of both is unknown. Clinical findings are similar to those of lymphedema praecox but may be less severe. Hereditary lymphedema type II (Meige disease, lymphedema praecox) develops around puberty or shortly thereafter in most individuals. This is the most common type of primary lymphedema. In addition to lymphedema of the legs, other areas of the body such as the arms, face and larynx may be affected. Some individuals may develop yellow nails. Lymphedema is prominent in some other genetic syndromes, including Turner syndrome; yellow nail syndrome, characterized by pleural effusions, chronic lung disease, lymphedema and yellow nails; and Hennekam syndrome, a rare congenital syndrome of generalized lymphatic abnormality, facial anomalies, and intellectual disability. The methods and compositions of the invention can be used to treat lymphatic dysfunction-associated abnormality of subcutaneous tissue or lymphatic disease of the subcutaneous tissue in a patient suffering from any of these primary lymphedemas and their symptoms.

Secondary (acquired) lymphedema is far more common than primary lymphedema. It is commonly caused by surgery (especially lymph node dissection, typically for staging and treatment of cancers), radiation therapy (especially axillary or inguinal), trauma, lymphatic obstruction by a tumor, increased white blood cells due to leukemia, infection, venous hypertension (phlebolymphedema) and, in developing countries, lymphatic filariasis, or any condition or treatment that limits or decreases lymph flow. In certain aspects, the methods described herein are used to treat secondary lymphedema. In additional aspects, the methods described herein are used to treat secondary lymphedema in a cancer patient (e.g., a patient being treated for that has been treated for cancer). In yet further aspect, the methods described herein are used to treat secondary lymphedema resulting from a non-cancer cause (e.g., in a patient that is not being treated for and that has not been treated for cancer).

In certain embodiments, the methods comprise treatment of lymphatic dysfunction-associated abnormality of subcutaneous tissue or lymphatic disease of the subcutaneous tissue in patients with established secondary lymphedema, for example, contracted as a result of cancer therapy. It has been estimated that more than 15% of breast cancer survivors experience secondary lymphedema. Surgical removal of lymph nodes or therapeutic radiation of lymph nodes increases the risk of lymphedema. After axillary intervention, 15% to 30% of breast cancer survivors experience clinically relevant lymphedema, but other types of cancer and their associated treatments may cause secondary lymphedema as well. The incidences of lymphedema associated with other malignancies (cancers) were as follows: soft tissue sarcoma 30%, lower extremity melanoma 28%, gynecologic cancer 20%, genitourinary cancer 10%, and head and neck cancers 50%. Lymphedema can also result from increased lymph production in patients with chronic venous insufficiency, congestive heart failure, and other causes of venous hypertension. The methods of the invention are applicable to the treatment of lymphatic dysfunction-associated abnormality of subcutaneous tissue or lymphatic disease of the subcutaneous tissue in any of such secondary lymphedema patients. In certain aspects, the lymphedema is upper limb extremity lymphedema. In an additional aspect, the patient developed upper limb extremity lymphedema after breast cancer treatment (referred to herein as breast cancer treatment associated upper limb lymphedema). In further aspects, the lymphedema is associated with head and neck cancer. In yet additional aspects, the lymphedema is lower limb lymphedema. In yet further aspects, the lymphedema is head, neck and trunk lymphedema.

The cardinal sign of acquired lymphedema is soft-tissue edema, graded in 4 stages. The term “established lymphedema” can refer generically to any of stages 1-3 of the disease, including without limitation the more advanced stages of the disease, e.g., stage 2 and stage 3, where structural changes in affected tissue are observed. In stage 0, the affected region is physically normal, but lymphatic insufficiency can be demonstrated through clinical assessment. In stage 1, the edema is pitting, and the affected area often returns to normal after elevation of the affected limb(s). In stage 2, the edema is pitting, and chronic soft-tissue inflammation causes structural changes in the tissues that accompany the pitting edema. In stage 3, the edema is brawny and irreversible, largely because of chronic soft-tissue structural changes.

In patients undergoing surgery and/or at risk of developing lymphedema, the treatment can be initiated (e.g., the initial dose can be administered), for example, immediately, or within one week, or one to three days after surgery. The treatment can also be initiated after surgical wound healing is complete, or immediately, or within one week. In additional aspects, treatment is initiated one to three days of initiation of radiotherapy, or cancer treatment, or at a time point before the onset or diagnosis of stage 0 lymphedema. The treatment can also be initiated after the onset or diagnosis of stage 0 lymphedema or after the onset or diagnosis of clinically evident lymphedema.

In a patient suffering from lymphedema, treatment with acebilustat can also reduce pain in patients with lymphedema. In addition to swelling, lymphedema is associated with localized pain (referred to herein as “lymphedema pain.” This pain has also been described as tenderness and/or discomfort. The term “lymphedema pain” is inclusive of tenderness and discomfort, for example, pain/tenderness/discomfort in one or both arms and/or pain/tenderness/discomfort in one or both legs. In certain embodiments, treatment with acebilustat as described herein reduces lymphedema pain. For example, lymphedema pain is reduced in the affected area after administration of acebilustat for six months or less, or three months or less. In certain aspects, pain is reduced by at least about 5%, 10%, 15%, 20% after administration of acebilustat for six months or less, or three months or less. Lymphedema pain can be measured, for example, by subjective pain scores as described, for example, in Smoot et al. (2015), J Cancer Surviv. 9: 287-304; the contents of which are expressly incorporated by reference herein.

As discussed herein, lymphedema and lipedema result in tissue changes that are visible by ultrasound. One such change is the appearance of “cobblestoning” or cobblestone appearance of the tissue. In certain embodiments, treatment with acebilustat as described herein improves or reduces the cobblestone appearance visible by ultrasound.

In certain aspects, treatment with acebilustat results in ultrasonographic changes in the affected (lymphedematous) area or tissue as compared to that at baseline. Lymphedema in some patients is associated with decreased visualization of the facia bands/planes of the affected subcutaneous tissue (e.g., the subcutaneous adipose tissue) under ultrasound and/or changes in echogenicity of the dermal and/or subcutaneous tissues. Treatment with acebilustat can result in a decrease in the subcutaneous echogenicity (for example, the echogenicity of the subcutaneous adipose tissue or a region of interest) of the affected (lymphedematous) area or tissue as compared to baseline. In additional aspects, the treatment described herein results in an improvement and/or visualization by ultrasound of the fascia bands of the subcutaneous adipose tissue of the affected area. In certain aspects, the dermal echogenicity to subcutaneous echogenicity ratio is increased after treatment as compared to that at baseline.

The invention also includes a method of treating lymphedema in a patient wherein the subcutaneous echogenicity of the affected area or tissue of the patient is increased as compared to normal, the method comprising administering to the patient an effective amount of acebilustat. “Normal” echogenicity in this context means the echogenicity of the same part of the anatomy in an individual (or individuals) not suffering from lymphedema and/or the echogenicity of the area (such as, a limb) or tissue contralateral to the affected area (or limb) or tissue. For example, if the patient is suffering from lymphedema in their right leg, “normal” echogenicity can be determined by ultrasound of their unaffected (non-lymphedematous) left leg.

The invention is also directed to a method of treating lymphedema in a patient, wherein the affected area or tissue of the patient has a dermal echogenicity to subcutaneous echogenicity ratio that is less than a control ratio, the method comprising administering to the patient an effective amount of acebilustat, and wherein echogenicity is determined by ultrasound. The “control ratio” is the control ratio is the mean dermal echogenicity to subcutaneous echogenicity ratio of a population of normal individuals or wherein the control ratio is the dermal echogenicity to subcutaneous echogenicity ratio of patient's tissue contralateral to the affected (lymphedematous) area or tissue. In certain aspects, the acebilustat is administered to said patient if the ratio is at least 5%, 10%, 15% or 20% lower than the control ratio.

Also encompassed is a method of treating lymphedema in a patient in need thereof, the method comprising:
determining dermal echogenicity and subcutaneous echogenicity of the affected area or tissue by ultrasound at baseline and calculating the ratio of dermal echogenicity to subcutaneous echogenicity at baseline;
administering acebilustat to said patient at an initial daily dose; and
determining dermal echogenicity and subcutaneous echogenicity of the affected area or tissue by ultrasound after treatment and calculating the ratio of dermal echogenicity to subcutaneous echogenicity after treatment;
continuing administration of acebilustat at the initial daily dose if the ratio after treatment is greater than the ratio at baseline, or modifying the dose of acebilustat if the ratio after treatment is not greater than the ratio at baseline. The dermal echogenicity and the subcutaneous echogenicity are determined at least about one month, at least about two months, at least about three months, at least about four months, at least about five months, at least about six months, at least about nine months, at least about one year, at least about 18 months or at least about 2 years after initiating administration of acebilustat. If the ratio is not increased (at the time point at which it is measured, e.g., 6 months after initiating the treatment), then the dose regimen can be modified, for example, the total daily dose can be increased.

In other embodiments, the invention is directed to a method of treating lymphatic dysfunction-associated abnormality of subcutaneous tissue or lymphatic disease of the subcutaneous tissue, wherein the patient has lymphedema (stage 0 to 3) as a result of cancer therapy, e.g., surgery, or radiotherapy, or other therapy or treatment damaging to the lymphatic system, wherein the method comprises administering an effective amount of acebilustat. In some embodiments, the individual has been treated with surgery, typically as a result of cancer diagnosis and treatment. In addition, other surgeries affecting the lymph nodes can cause lymphedema treatable in accordance with the invention. In such embodiments, treatment with acebilustat can be initiated before surgery, immediately following surgical wound healing, or can be initiated at a time following surgical wound healing, including after some or even substantial wound healing has occurred, e.g., 3-14 days after surgery, but before a patient has been diagnosed as having stage 0 lymphedema. In yet additional aspects, the patient has been treated with radiotherapy, which itself may follow surgery for cancer therapy. In such embodiments, treatment with acebilustat can be initiated before radiotherapy, immediately during radiotherapy, following radiotherapy, or may commence at a time following radiotherapy, including after some or even substantial wound healing has occurred, as above, but before a patient has been diagnosed as having stage 0 lymphedema.

The invention encompasses a method of treating lymphatic dysfunction-associated abnormality of subcutaneous tissue, lymphatic disease of the subcutaneous tissue, or lymphedema in a patient suffering therefrom wherein the patient has undergone lymph node dissection or radiation therapy, for example, as part of cancer treatment, the method comprising administering an effective amount of acebilustat.

The invention also includes a method of treating lymphatic dysfunction-associated abnormality of subcutaneous tissue or lymphatic disease of the subcutaneous tissue in patient in need thereof, wherein the patient is suffering from upper extremity lymphedema (e.g. lymphedema in an arm), e.g., after cancer surgery and/or radiation therapy, wherein the method comprises administering to said patient an effective amount of acebilustat. For example, the patient can be suffering from upper limb extremity lymphedema in a patient after breast cancer surgery and/or radiation therapy. Arm or upper limb lymphedema can be caused by interruption of the axillary lymphatic system by surgery or radiation therapy, resulting in accumulation of fluid in subcutaneous tissue in the arm.

The invention additionally includes a method of treating lymphatic dysfunction-associated abnormality of subcutaneous tissue or lymphatic disease of the subcutaneous tissue in patient in need thereof, wherein the patient is suffering from lower limb lymphedema (e.g., lymphedema in a leg). In certain examples, the patient suffering from lower limb lymphedema is not a cancer patient.

In some embodiments, the invention is a method of treating lymphatic dysfunction-associated abnormality of subcutaneous tissue, lymphatic disease of the subcutaneous tissue, or lymphedema in a patient in need thereof (e.g., after lymph node dissection) comprising administering an effective amount of acebilustat in combination with a surgical intervention intended to treat or ameliorate the condition. Non-limiting examples of surgical interventions used to treat lymphedema are lymphovenous anastomosis (LVA) (also referred to in the literature as lymphovenous bypass or LVB), vascularized lymph node transfer (VLNT), and implantation of an artificial lymph node. LVA is used to treat lymphedema and improves physical symptoms and quality of life (Jonis et al. (2024), Nature Scientific Reports 14: 2238). In certain aspects, the invention is a method of treating lymphatic dysfunction-associated abnormality of subcutaneous tissue, lymphatic disease of the subcutaneous tissue, or lymphedema comprising administering acebilustat in combination with LVA. An artificial lymph node is an implantable device designed to maintain lymph flow after lymph node removal surgery (see, for example, Duran-Aguilar et al. (2022), Healthcare (Basel) 10(1): 68; U.S. Pat. Nos. 8,101,195, 9,234,175, 8,030,070, JP 2004255110A, JP2012036151A, US2006171988A and JP2006129839A). In certain aspects, the invention is a method of treating lymphatic dysfunction-associated abnormality of subcutaneous tissue, lymphatic disease of the subcutaneous tissue, or lymphedema comprising administering acebilustat in a patient implanted with an artificial lymph node. VLNT involves the transfer of functional lymph nodes to an area of the body with damaged or diseased lymphatic drainage. In certain aspects, the invention is a method of treating lymphedema comprising administering acebilustat in combination with VLNT.

The invention additionally encompasses a method of preventing or lowering the risk of lymphatic dysfunction-associated abnormality of subcutaneous tissue, lymphatic disease of the subcutaneous tissue, or lymphedema in a patient in need thereof comprising administering to said patient an effective amount of acebilustat. A patient in need of preventing or lowering the risk of lymphatic dysfunction-associated abnormality of subcutaneous tissue, lymphatic disease of the subcutaneous tissue, or lymphedema is a patient that is at risk of developing lymphedema, for example, a patient that has undergone surgery (e.g., lymph node dissection, typically for staging and treatment of cancers), radiation therapy (especially axillary or inguinal), trauma, lymphatic obstruction by a tumor, increased white blood cells due to leukemia, infection, venous hypertension (phlebolymphedema), filariasis (in developing countries), or any condition or treatment that limits or decreases lymph flow. In certain aspects, the invention is a method of preventing or lowering the risk of lymphatic dysfunction-associated abnormality of subcutaneous tissue, lymphatic disease of the subcutaneous tissue, or lymphedema in a patient undergoing or that has undergone lymph node dissection or radiation therapy, for example, as part of cancer treatment. The method comprises administering acebilustat after the surgery or radiation therapy and optionally, administering acebilustat prior to the surgery or initiation of radiation therapy. With respect to surgery, lymphatic dysfunction-associated abnormality of subcutaneous tissue, lymphatic disease of the subcutaneous tissue, or lymphedema can, for example, develop in a delayed manner one or more years after surgery, for example one to five years after surgery (Cemal et al. (2013), J Am Coll Surg 213(4): 543-551. The method can comprise administration of acebilustat after surgery (e.g., lymph node dissection), for example, within hours or a week after surgery, and administration of acebilustat can be continued until the risk of lymphatic dysfunction-associated abnormality of subcutaneous tissue, lymphatic disease of the subcutaneous tissue, or lymphedema is significantly decreased, for example, the acebilustat can be administered for at least six months, at least one year, at least eighteen months, at least two year, at least 30 months, at least three years, at least four years, at least five years, at least six years, or more after surgery. The method can optionally also comprise administration of acebilustat prior to the surgery. The risk of developing lymphatic dysfunction-associated abnormality of subcutaneous tissue, lymphatic disease of the subcutaneous tissue, or lymphedema by treatment with acebilustat can be reduced as compared to that in the absence of acebilustat treatment. In certain aspects, administration of acebilustat after surgery reduces the risk of developing lymphedema by at least about 5%, 10%, 15%, 20%, 25%, 30%, 40%, 45%, or 50% or more, as compared to that in the absence of acebilustat treatment. The methods of preventing or lower the risk of lymphatic dysfunction-associated abnormality of subcutaneous tissue, lymphatic disease of the subcutaneous tissue, or lymphedema encompass reducing the severity of the lymphatic dysfunction-associated abnormality of the SAT, lymphatic disease of the SAT, or lymphedema, or symptoms of either of thereof. For example, the methods encompass reducing the severity of lymphedema that develop as compared to the patient's risk in the absence of acebilustat administration. For example, the methods encompass reducing the risk that the patient develops Stage 1, 2 or 3 lymphedema.

In some embodiments, the invention is a method of preventing or lowering the risk of lymphatic dysfunction-associated abnormality of subcutaneous tissue, lymphatic disease of the subcutaneous tissue, or lymphedema in a patient in need thereof comprising administering an effective amount of acebilustat (e.g., after lymph node dissection) in combination with another prophylactic intervention including, for example, pharmacologic treatment or prophylactic surgical intervention, wherein the method comprises administering an effective amount of acebilustat. For example, the method can prevent or lower the risk of lymphatic dysfunction-associated abnormality of subcutaneous tissue, lymphatic disease of the subcutaneous tissue, or lymphedema in a patient after surgery (e.g., lymph node dissection) comprising administration of acebilustat and the additional prophylactic intervention after the surgery. Non-limiting examples of prophylactic surgical interventions are lymphovenous anastomosis (LVA) (also referred to in the literature as lymphovenous bypass or LVB), and implantation of an artificial lymph node. LVA is used prophylactically to reduce the risk of lymphedema in cancer patients (Deldar et al. (2023), Ann Surg Open 4(2): e278). In certain aspects, administering acebilustat in addition to prophylactic LVA can reduce the risk of developing lymphedema as compared to that in the absence of the acebilustat treatment. An artificial lymph node is an implantable device designed to maintain lymph flow after lymph node removal surgery (see, for example, Duran-Aguilar et al. (2022), Healthcare (Basel) 10(1): 68; U.S. Pat. Nos. 8,101,195, 9,234,175, 8,030,070). In certain aspects, the administration of acebilustat in combination with implantation of an artificial lymph node can reduce the risk of developing lymphedema as compared to that in the absence of the acebilustat treatment.

In patients at risk of lymphedema due to surgery, radiotherapy or cancer treatment, the treatment can be initiated (e.g., the initial dose can be administered), for example, immediately, or within one week, or one to three days after the surgery, radiotherapy or cancer treatment is initiated or can be initiated before the surgery, radiotherapy or cancer treatment is initiated. The treatment can also be initiated after surgical wound healing is complete, or immediately, or within one week, or one to three days of initiation of radiotherapy, or cancer treatment. In certain aspects, administration of acebilustat is administered prior to the surgery and continues after the surgery as described above.

The invention also includes a method of preventing or inhibiting progression of lymphedema in an individual to be treated that has, or that has been, or is being treated for cancer but has not yet developed lymphedema. The invention can also be practiced in other prophylactic modes, including after successful treatment. Some patients can be treated in accordance with the invention and obtain complete or significantly complete recovery and not benefit from further treatment. Other patients, however, can benefit from continued administration of acebilustat or other selective LTA4H inhibitor after treatment has been successful to prevent recurrence of the disease. Thus, for prophylactic purposes of lymphedema prevention, following treatment by the methods of the invention, the architecture of the skin of the affected patient, e.g., skin of the extremities, remains with a substantially normal architecture consistent with successful treatment. Following prophylactic treatment with the methods of the invention, the volume of tissue, e.g., upper extremities, lower extremities, etc. should be stable over time, relative to a control group in the absence of treatment. The time period in which to see treatment benefit can be about 1 week, about 2 weeks, about 3 weeks, about 1 month, about 2 months, about 3 months, about 4 months, or more. In some embodiments, the volume and/or structure of the affected or at risk tissue, i.e., the lymphedematous tissue, is measured or otherwise assayed or assessed at various points over the time period, e.g., at least at the beginning and some designated endpoint for testing. In some embodiments, the architecture of the affected tissue is assayed by dermal thickness measurements or by histological assessment; absence of lymphedema can be ascertained by serial measurement of limb bioimpedance. The effect on the lymphatic system can also be assessed using lymphoscintigraphy and/or indocyanine green lymphography, for example. In some embodiments, the architecture of the affected tissue after treatment resembles or more closely resembles the architecture of unaffected tissue.

The invention also includes a method of promoting lymphangiogenesis and/or lymphatic repair in a patient in need thereof comprising administering to said patient an effective of acebilustat. Such patients include patients at risk for at risk of lymphatic dysfunction-associated abnormality of subcutaneous tissue, lymphatic disease of the subcutaneous tissue, or lymphedema including, for example, a patient that has undergone surgery (e.g., lymph node dissection, typically for staging and treatment of cancers), radiation therapy (especially axillary or inguinal), trauma, lymphatic obstruction by a tumor, increased white blood cells due to leukemia, infection, venous hypertension (phlebolymphedema), filariasis (in developing countries), or any condition or treatment that limits or decreases lymph flow. In certain aspects, the invention is a method of promoting lymphangiogenesis and/or lymphatic repair in a patient at risk of developing lymphedema, wherein the patient undergoing or that has undergone node dissection or radiation therapy. In yet additional aspects, the patient is implanted with an artificial lymph node; in certain aspects, treatment with acebilustat increases lymphatic growth and repair to and from the artificial lymph node. In yet additional aspects, the invention is a method of promoting lymphangiogenesis and/or lymphatic repair to an artificial lymph node in a patient implanted with the artificial lymph node, the method comprising administering to said patient an effective of acebilustat. As described above, the treatment can be initiated (e.g., the initial dose can be administered), for example, immediately, or within one week, or one to three days after the surgery, radiotherapy or cancer treatment. The treatment can also be initiated after surgical wound healing is complete, or immediately, or within one week, or one to three days of initiation of radiotherapy, or cancer treatment. In certain aspects, administration of acebilustat is administered prior to the surgery and continues after the surgery as described above. In yet additional aspects, the methods further comprise administration of retinoic acid. Although not wishing to be bound by theory, acebilustat promotes lymphangiogenesis by reducing inflammation and/or enhancing Notch signaling and VEGFR3 expression. By lowering LTB4, acebilustat can allow for the resumption of normal signaling for both these pathways.

Acebilustat and other LTA4H inhibitors have been described, for example, in U.S. Pat. Nos. 7,737,145, 9,820,974, and U.S. Patent Application Publication No. 20100210630A1, the contents of each of which are incorporated by reference herein. The chemical name of acebilustat is 4-{[(1S,4S)-5-({4-[4-oxazol-2-yl-phenoxy]phenyl}methyl)-2,5-diazabicyclo[2.2.1]heptan-2-yl]methyl}benzoic acid (also referred to as CTX-4430). Acebilustat is a potent inhibitor of Leukotriene A4 Hydrolase (LTA4H), the rate-limiting enzyme in production of leukotriene B4 (LTB4).

The methods of the invention comprise administration of an effective dose of acebilustat (also known as CTX-4430) to human patients. In certain aspects, the acebilustat is administered orally. This compound and methods for the preparation thereof have been described in detail in U.S. Pat. Nos. 7,737,145, 9,820,974, and U.S. Patent Application Publication No. 20100210630A1, the contents of each of which are incorporated by reference herein. Acebilustat has the chemical structure shown below:

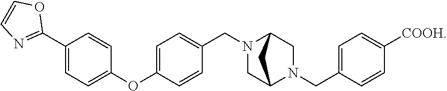 [SMILES: O=C(O)c1ccc(CN2C[C@@H]3C[C@H]2CN3Cc2ccc(Oc3ccc(-c4ncco4)cc3)cc2)cc1]

In vitro, acebilustat inhibits the epoxide hydrolase enzymatic activity of LTA4H with an IC₅₀ of 6.3 ng/mL for LTB4 production. In human whole blood tested ex vivo, acebilustat inhibits LTB4 production with an approximate IC₅₀ of 30.8 ng/mL. Acebilustat 48 ng/mL has also been shown to reduce neutrophil swarming in vitro by 80% in response to factors present in human cystic fibrosis (CF) sputum. In pharmacodynamic studies in humans, acebilustat inhibits LTB4 production with an estimated in vivo EC₅₀ of 93 ng/mL. In CF patients, sputum white blood cells were decreased by 31% from Baseline in all treated subjects (doses of 50 or 100 mg) and by 60% from Baseline in the 100 mg acebilustat group. Sputum neutrophils decreased by 34% in all treated patients (doses of 50 to 100 mg) and by 65% in the 100 mg group. In a recent study of Phase II adult patients with CF, acebilustat showed promise in reducing the rate of pulmonary exacerbations over the course of 48 weeks of treatment with no evidence of increased risk of infection (described, for example, in U.S. Pat. No. 10,898,484; the contents of which are expressly incorporated by reference herein). This effect was most notable in patients with early disease.

In some examples, an effective amount of acebilustat administered orally can be about 200 mg or less. The invention thus encompasses oral administration of about 200 mg or less acebilustat to the patient. In certain aspects, the patient is administered about 200 mg of acebilustat; for example, chronic oral administration (e.g., for more than about one day, for at least about one week, for at least about two weeks, for at least about three weeks, for at least about one month, for at least about 2 months, for at least about 3 months, for at least about 4 months, for at least about 5 months, or for at least about 6 months or 24 weeks, and/or throughout the patient's treatment). The invention encompasses oral administration of about 100 mg acebilustat to said patient; for example, chronic oral administration (e.g., for more than about one day, for at least about one week, for at least about two weeks, for at least about three weeks, for at least about one month, for at least about 2 months, for at least about 3 months, for at least about 4 months, for at least about 5 months, or for at least about 6 months or 24 weeks, and/or throughout the patient's treatment). The invention also encompasses administration of about 50 mg acebilustat to said patient; for example, chronic oral administration (e.g., for at least about one week, for at least about two weeks, for at least about three weeks, for at least about one month, for at least about 2 months, for at least about 3 months, for at least about 4 months, for at least about 5 months, or for at least about 6 months or about 24 weeks, and/or throughout the patient's treatment). Acebilustat can, for example, be administered at a dose of about 50 mg every 12 or 24 hours (or once or twice a day), or at a dose of about 100 mg every 12 or 24 hours (or once or twice a day). In certain aspects, acebilustat is administered at a dose of about 100 mg every 24 hours (or once a day). The total daily dose of acebilustat can be a dose that is about 200 mg or less, about 100 mg or less, or about 50 mg or less. The total daily dose of acebilustat can also be from about 100 mg to about 200 mg, for example about 150 mg. The total daily dose of acebilustat can also be from about 50 mg to about 100 mg, for example, about 75 mg. In certain aspects, the dose of acebilustat is about 25 mg administered once or twice a day or a dose between about 25 and about 50 mg administered once or twice a day. Acebilustat can be administered with or without food.

In certain aspects, an effective amount of acebilustat or other selective LTA4H hydrolase inhibitor is administered in an effective amount, wherein the effective amount is less than that which provides maximum inhibition of LTA4H epoxide hydrolase activity. In some examples, the effective amount can provide at least about 50%, at least about 60%, at least about 70%, at least about 80%, or at least about 85% inhibition of LTA4H epoxide hydrolase activity, e.g., at least about 50%, at least about 60%, at least about 70%, at least about 80%, or at least about 85% inhibition of LTB4 generation. It has been shown, for example, that a 50 mg dose of acebilustat provides greater than or equal to 63% LTB4 inhibition, that a 100 mg dose of acebilustat provides greater than or equal to about 74% LTB4 inhibition, that a 150 mg dose provides greater than or equal to about 82% LTB4 inhibition and that 200 mg acebilustat provides greater than or equal to about 85% LTB4 inhibition in human pharmacodynamic studies.

In further aspects, the acebilustat or other selective LTA4 hydrolase inhibitor is administered topically.

The invention also includes methods wherein the acebilustat or other selective LTA4 hydrolase inhibitor is administered by pulsatile dosing or wherein the acebilustat is in a pulsatile release pharmaceutical composition. Pulsatile dosing encompasses administration or release of an active agent or drug after a pre-determined off-released period or lag time. As such, pulsatile dosing can include delivering a drug rapidly and completely after a period of no drug release. For example, the concentration of the active agent or drug in the plasma is allowed to drop below about 50% inhibition of the LTA4H activity or below about 50% of LTB4 generation before the next dose is administered. The acebilustat or other selective LTA4H inhibitor can also be administered in a controlled release, sustained release manner, and/or delayed release manner. The acebilustat or other selective LTA4H inhibitor can also be administered in a controlled release formulation, including, for example, a sustained release formulation and/or a delayed release formulation. “Controlled release” refers to a drug-containing formulation or unit dose form thereof from which release of the drug is not immediate, i.e., with a controlled release formulation, administration does not result in immediate release of all of the drug administered into an absorption pool. The term is used interchangeably with “nonimmediate release” as defined in Remington: The Science and Practice of Pharmacy, Nineteenth Ed. (Easton, Pa.: Mack Publishing Company, 1995). In general, controlled release formulations include sustained release and delayed release formulations. “Sustained release” and “extended release” means a drug formulation that provides for gradual release of a drug over an extended period of time, and typically, although not necessarily, results in substantially constant blood levels of a drug over an extended time period. “Delayed release” refers to a drug formulation that, following administration to a patient, provides a measurable time delay before drug is released from the formulation into the patient's body.

The treatment can include administration of the same dose and/or dosing regimen throughout the patient's treatment. The method can also include an initial dose(s) of the acebilustat or other selective LTA4H inhibitor followed by maintenance therapy comprising one or more maintenance doses, wherein the one or more maintenances doses are different from the initial dose. For example, the one or more maintenance doses can be lower than the initial dose and/or can be less frequent than the initial dosing regimen. In another example, the one or more maintenance doses can be higher than the initial dose and/or can be more frequent than the initial dosing regimen.

The invention encompasses methods of treating lymphatic-dysfunction associated abnormality of the subcutaneous tissue or lymphatic disease in the subcutaneous tissue using a selective LTA4H inhibitor other than acebilustat. The invention also encompasses a method of treating lipedema comprising administering a selective LTA4H inhibitor other than acebilustat. LTA4H is an epoxide hydrolase that generates LTB4 from LTA4. The invention further encompasses a method of preventing or reducing the risk of lymphatic dysfunction-associated abnormality of subcutaneous tissue, lymphatic disease of the subcutaneous tissue, or lymphedema in a patient in need thereof comprising administering a selective LTA4H inhibitor other than acebilustat. LTB4 is a pro-inflammatory mediator and thus inhibition of LTA4H activity inhibits the production of the pro-inflammatory LTB4. LTA4H also is an aminopeptidase and degrades the tripeptide Pro-Gly-Pro (PGP) which is a neutrophil chemoattractant (Low et al. (2017), Scientific Reports 7, 44449 (2017). https://doi.org/10.1038/srep44449; the contents of which are expressly incorporated by reference herein). The accumulation of PGP is associated with pro-inflammatory effects. Thus, LTA4H plays an important anti-inflammatory role in degrading PGP, which role is paradoxical to its pro-inflammatory epoxide hydrolase activity. The present invention is at least partially based on the discovery that treatment with acebilustat treatment resulted skin and adipose tissue changes, including, for example, ultrasonographic changes in the affected area. A selective LTA4H inhibitor is an agent or compound that has more selectivity for LTA4H than other aminopeptidase enzymes and/or an agent that has more selectivity for the epoxide hydrolase activity of LTA4H than the aminopeptidase activity of LTA4H. In the context of LTA4 inhibitors, selectivity refers to potency of inhibition. For example, an LTA4H inhibitor is more selective for the epoxide hydrolase activity of LTA4H than the aminopeptidase activity of LTA4H when the IC₅₀ for epoxide hydrolase inhibition is lower than the IC₅₀ for aminopeptidase activity. In another example, an LTA4H inhibitor is more selective for inhibiting LTA4H than other aminopeptidases when the IC₅₀ for LTA4H inhibitor is lower than the IC₅₀ for other aminopeptidases. A selective LTA4H inhibitor that is more selective for the epoxide hydrolase activity of LTA4H than the aminopeptidase activity of LTA4H inhibits generation of LTB4 while having minimal effect on PGP degradation. Acebilustat is a selective LTA4H inhibitor that is more selective for the epoxide hydrolase activity of LTA4H than the aminopeptidase activity of LTA4H and is more selective for LTA4H inhibition than aminopeptidase inhibition. Specifically, acebilustat has a 2-fold preference for inhibiting epoxide hydrolase activity versus aminopeptidase activity (Bhatt et al. (2017), Seminars in Immunology 33: 65-73; the contents of which are expressly incorporated by reference herein). Specifically, Bhatt et al. teaches that acebilustat shows 2-fold functional selectivity for LTA4H epoxide hydrolase (12 nM) versus LTA4H aminopeptidase (27 nM) and is highly selective for LTA4H versus other metalloenzymes. In contrast, some other LTA4H inhibitors, such as SC567461A (Searle/Pharmacia), DG-051 (DeCODE Pharmaceuticals), and JNJ-40929837 (Johnson and Johnson), have similar potencies at inhibiting LTB4 generation and PGP degradation and thus may have a pro-inflammatory effect by resulting in PGP accumulation. Such inhibitors are referred to herein as “non-selective LTA4H inhibitors.” Ubenimex is a non-selective LTA4H inhibitor and is a broad spectrum aminopeptidase inhibitor (Bhatt et al. (2017); Inoi et al. (1995), Anticancer Res. 15(5B): 2081-2087; the contents of which are expressly incorporated by reference herein).

The IC₅₀ of LTA4H epoxide hydrolase activity can be measured using methods known in the art, for example, using the LTA4 hydrolase homogenous time resolved fluorescence assay which is described, for example, in U.S. Pat. Nos. 7,737,145 and 10,202,362; the contents of which are expressly incorporated by reference herein. The hydrolase-homogeneous time resolved fluorescence assay is a two-step assay that measures the hydrolysis of LTA₄ to LTB₄ by analyzing the amount of LTB₄ produced. The first step involves the enzymatic conversion of LTA₄ to LTB₄ and the second step involves the quantification of the LTB₄ formed with a homogeneous time resolved fluorescence assay. LTA4H epoxide hydrolase activity can also be measured in a whole blood assay, for example, using human whole blood, using the method described in Penning, T. D. et al., J. Med. Chem. (2000), 43(4): 721-735 and U.S. Pat. No. 7,737,145; the contents of each of which are expressly incorporated by reference herein. In the whole blood assay, the inhibitor compounds are tested for their ability to inhibit LTB₄ release upon stimulation with calcium ionophore and the LTB₄ levels in supernatants are measured by ELISA. LTA4H epoxide hydrolase activity can also be measured according to the method described in Low et al. (2017). The IC₅₀ for LTA4H aminopeptidase activity can be measured using methods known in the art, for example, using the method described in Kull et al., The Journal of Biological Chemistry 274(49): 34683-34690, the method described in U.S. Pat. No. 10,202,362, and/or the method described in Low et al. (2017); the contents of each of which are expressly incorporated by reference herein. Other methods for measuring LTA4H epoxide hydrolase activity and/or LTA4H peptidase activity are described, for example, in Askonas, L. J., et al., The Journal of Pharmacology and Experimental Therapeutics 2002, 300(2): 577-582; Penning, T. D., J. Med. Chem. 2000, 43(4): 721-735; Kull, F. et al., The Journal of Biological Chemistry 1999, 274 (49): 34683-34690; the contents of which are expressly incorporated by reference herein.

The invention includes a method of treating lymphatic-dysfunction associated abnormality of the subcutaneous tissue or lymphatic disease in the subcutaneous tissue comprising administering to a patient in need thereof an effective amount of a selective LTA4H inhibitor. The present invention also encompasses a method of treating lipedema comprising administering to a patient in need thereof an effective amount of a selective LTA4H inhibitor. The invention further encompasses a method of preventing or reducing the risk of lymphatic dysfunction-associated abnormality of subcutaneous tissue, lymphatic disease of the subcutaneous tissue, or lymphedema in a patient in need thereof comprising administering a selective LTA4H inhibitor other than acebilustat. The selective LTA4H inhibitor can, for example, have at least about 1.5 times or at least about 2 times more selectivity for the epoxide hydrolase activity of LTA4H versus the aminopeptidase activity of LTA4H. The selective LTA4H inhibitor can additionally or alternatively, have at least about 1.5 times, at least about 2 times, at least about 2.5 times at least about 3 times, at least about 5 times, or at least about 10 times more selectivity for LTA4H than other aminopeptidases. In yet another embodiment, the LTA4H inhibitor is at least about 2 times more selective for LTA4H than other aminopeptidases and more than about 1.5 times more selective for epoxide hydrolase activity than aminopeptidase activity of LTA4H. The LTA4H inhibitor can also be at least about 2.5 times, at least about 3 times, at least about 5 times, or at least about 10 times more selective for epoxide hydrolase activity than aminopeptidase activity of LTA4H. In yet a further embodiment, the LTA4H inhibitor is at least about 2 times more selective for LTA4H than other aminopeptidases and more than about 2 times more selective for epoxide hydrolase activity than aminopeptidase activity of LTA4H. The selective LTA4H inhibitors include, for example, acebilustat. Other selective LTA4H inhibitors have been described in Low et al. and include compounds with a resveratrol core as described therein. Non-limiting examples of LTA4H inhibitors include, for example, cis-resveratrol, trans-resveratrol, isoflavone daidzein, and 7,8,4′-trihydroxyisoflavone. Additional, non-limiting examples of LTA4H inhibitors are shown in the Tables below:

TABLE 1

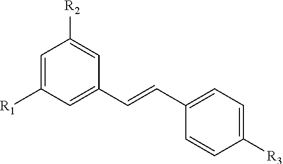

| Cmpd | | | | EH IC50 ± | Aminopeptidase |
|---|---|---|---|---|---|
| No. | R1 | R2 | R3 | SEM (uM) | IC50 ± SEM (uM) |
| 1 | OH | OH | OH | 32.2 ± 5.9 | >100 |
| 2 | OH | OH | OH | 14.3 ± 2.1 | 85.3 ± 33.9 |
| (Z- | | | | | |
| isomer) | | | | | |
| 4 | OCOMe | OCOMe | OCOMe | 38.6 ± 5.9 | >100 |
| 5 | OMe | OH | OH | 3.9 ± 0.4 | 99.5 ± 22.6 |

TABLE 2

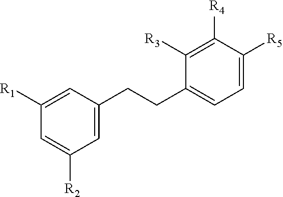

| | | | | | | EH IC50 ± | Aminopeptidase |
|---|---|---|---|---|---|---|---|
| Cmpd No. | R1 | R2 | R3 | R4 | R5 | SEM (uM) | IC50 ± SEM (uM) |
| 6 | OMe | OH | H | H | OH | 6.4 ± 0.7 | >100 |
| 7 | OEt | OH | H | H | OH | 3.2 ± 0.7 | >100 |
| 8 | OMe | OH | H | OH | H | 1.7 ± 0.25 | >100 |
| 9 | OMe | OH | OH | H | H | 0.5 ± 0.04 | >100 |

TABLE 3

Compound 10

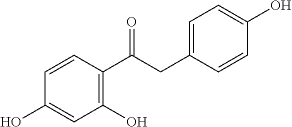 [SMILES: O=C(Cc1ccc(O)cc1)c1ccc(O)cc1O]

Isoflavones

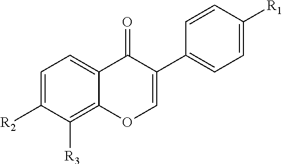

| | | | | Epoxide Hydrolase | Aminopeptidase |
|---|---|---|---|---|---|
| Cmpd. No. | R₁ | R₂ | R₃ | IC₅₀ ± SEM (μM) | IC₅₀ ± SEM (μM) |
| 10 | | | | 34.24 ± 4.7 | >100 |
| 11 | OH | OH | H | 15.0 ± 23 | >100 |
| 12 | OH | OH | OH | 7.3 ± 0.8 | 62.7 ± 13.3 |
| 13 | H | OH | H | 48.2 ± 14.6 | >100 |
| 14 | OH | O—CH₂—O | | 20.6 ± 2.4 | 20.6 ± 3.3 |
| 15 | OH | Me | OH | 1.2 ± 0.21 | 69.2 ± 10.9 |

In certain aspects, an effective dose of acebilustat is administered to an individual having lipedema, including without limitation, established lipedema, for a period of time sufficient to decrease or reverse tissue pathology of the affected (lipedematous) tissue relative to an untreated control group or relative to that at the patient's baseline. In some embodiments, an effective dose of acebilustatis administered to an individual having a lymphatic dysfunction-associated abnormality of subcutaneous tissue or lymphatic disease of the subcutaneous tissue for a period of time sufficient to decrease or reverse tissue pathology of the affected tissue relative to an untreated control group or relative to that at the patient's baseline. As described above, the decrease or reversal in tissue pathology can be assessed by ultrasonographic changes. Treatment can be continued as required for maintenance of the therapeutic benefit: where required, maintenance therapy can be maintained at the same dosage and schedule as previous treatment or may be achieved by transitioning to an alternative maintenance schedule, e.g., at a lower dose, less frequent dose, and the like. In some embodiments, the treating physician can determine that the treatment is efficacious by verifying a change in the architecture of the affected tissue. The tissue can be assayed by any number of means as described herein to verify therapeutic benefit, if visual inspection alone is insufficient. A measure of efficacy for lipedema and lymphedema is ultrasonographic changes of the affected (lipedematous) area and/or reduction in the amount of fat or adipose in the affected area and/or structural changes in the adipose tissue of the affected area. An additional measure of efficacy is an improvement in lymphatic symptoms, for example, as demonstrated by lymphoscintigraphy and indocyanine green (ICG) lymphography. Additional methods of determining if the treatment is efficacious include lymphoscintigraphy, magnetic resonance lymphangiography, and indocyanine green (ICG) lymphography.

In some embodiments, an effective dose of acebilustat or other selective LTA4H inhibitor is provided to an individual susceptible to lymphatic dysfunction-associated abnormality of subcutaneous tissue or lymphatic disease of the subcutaneous tissue. Such patients include without limitations individuals that have undergone surgery or radiation for cancer (these patients are at risk for secondary lymphedema). In further aspects, the lymphedema is secondary, or acquired lymphedema. Additionally, patients at risk for lymphatic dysfunction-associated abnormality of subcutaneous tissue or lymphatic disease of the subcutaneous tissue are female patients that have a family member with lipedema and/or is a patient that is a carrier for a gene associated with lipedema.

In the methods described herein, acebilustat can be administered to a patient on top of their current treatment regime, or on top of the standard of care. The standard of care for the treatment of lymphedema includes, but is not limited to, diuretics, antibiotics, exercise, manual lymph drainage, compression bandages, as well as compression garments, for example. The methods of the invention can also include administration of a therapeutically effective amount of at least one additional active agent other than a LTA4H inhibitor. The additional agent can, for example, be selected from the group consisting of a selective COX-1 inhibitor, a selective COX-2 inhibitor, a non-selective COX-1/COX-2 inhibitor, a coumarin, an anti-histamine, montelukast and other related leukotriene inhibitors, an anti-fibrotic compound, a diuretic, a statin, an mTOR inhibitor, and pirfenidone. In certain aspects, the additional agent can, for example, be selected from the group consisting of a selective COX-1 inhibitor, a selective COX-2 inhibitor, a non-selective COX-1/COX-2 inhibitor, a coumarin, an anti-histamine, an anti-fibrotic compound, a diuretic, a statin, an mTOR inhibitor, and pirfenidone. Non-limiting examples of non-selective COX-1/COX-2 inhibitors include salicylic acid derivatives including aspirin, sodium salicylate, choline magnesium trisalicylate, salsalate, diflunisal, sulfasalazine and olsalazine; para-aminophenol derivatives such as acetaminophen, indole and indene acetic acids such as indomethacin and sulindac, heteroaryl acetic acids including tolmetin, diclofenac and keterolac, arylpropionic acids including ibuprofen, naproxen, flurbiprofen, ketoprofen, fenoprofen and oxaprozin, anthranilic acids (fenamates) including mefanamic acid and meclofenamic acid, enolic acids including oxicams such as piroxicam and meloxicam and alkanones such as nabumetone, as well as pharmaceutically effective esters, salts, isomers, conjugates and prodrugs thereof. In yet additional aspects, the non-selective COX-1/COX-2 inhibitor is a propionic acid derivative, such as ketoprofen. Selective COX-2 inhibitors include, for example, celecoxib. Non-limiting examples of diuretics are furosemide, torasemide and hydrochlorothiazide, recombinant angiotensin converting enzyme-2 and acetylsalicylic acid. Statins include, for example, atorvastatin, fluvastatin, lovastatin, pitavastatin, pravastatin, rosuvastatin, simvastatin, and/or ezetimibe/simvastatin combination. mTOR inhibitors include, for example, rapamycin, everolimus, and sirolimus, and rapalog. Anti-fibrotic agents include the following non-limiting examples: pirfenidone, Transforming Growth Factor beta (TGFβ) inhibitors such as galunisertib, and Connective Tissue Growth Factor (CTGF) inhibitors such as FG-3019. The additional active agent can also be any other agent used in the treatment of lymphedema and/or pro-lymphangiogenic drugs like retinoic acids (including, for example, 9-cis retinoic acid). In another example, the additional active agent is an agent, e.g., a biologic or a small molecule that that targets and/or inhibits an anti-lymphangiogenic growth factor or cytokine including, but not limited to, anti-TGF-b1 antibodies, anti-IFN-γ antibodies, anti-IL-4 antibodies and anti-IL-13 antibodies. For example, the additional agent can be an antibody (e.g., a monoclonal antibody) that targets and/or inhibits an anti-lymphangiogenic growth factor or cytokine including, but not limited to, anti-TGF-b1 antibodies, anti-IFN-γ antibodies, anti-IL-4 antibodies and anti-IL-13 antibodies. The antibody can be one that targets one or more anti-lymphangiogenic growth factors or cytokines including, but not limited to, dupilumab (a monoclonal antibody that inhibits IL-4 and IL-13), and pascolizumab (a humanized anti-IL-4 monoclonal antibody). The additional agent can also be pitrakinra, a human recombinant protein that is an antagonist of IL-4 and IL-13. It is to be understood that when the acebilustat or other selective LTA4H inhibitor is co-administered or in combination with the at least one additional active agent, the acebilustat or other selective LTA4H inhibitor can be administered simultaneously with, prior to, or after administration of one or more additional active agents. For example, the initiation of acebilustat treatment can be subsequent to the initiation of treatment with the additional active agent. In another example, the initiation of treatment with the additional active agent can be subsequent to the initiation of acebilustat treatment. Such combination therapy includes administration of a single pharmaceutical dosage formulation which contains the acebilustat or other selective LTA4H inhibitor and the one or more additional active agents, as well as administration of the acebilustat and each active agent in its own separate pharmaceutical dosage formulation. Where separate dosage formulations are used, acebilustat and one or more additional active agents can be administered at essentially the same time, i.e., concurrently, or at separately staggered times, i.e., sequentially; and/or in the same treatment session and/or as part of the same treatment regimen; and/or daily administration of acebilustat and daily administration of the one or more additional active agents. Combination therapy and concomitant administration is understood to include all these regimens.

The acebilustat or other selective LTA4H inhibitor can continue to be administered if the therapy is determined to be efficacious. As described above, efficacy can be assessed by ultrasound. For example, in certain lymphedema patients, the subcutaneous echogenicity of the affected area is increased as compared to normal, there is blurring of (or a lack of contrast between) the interface between the subcutaneous fat and the skin, and/or a loss of distinctness of the fascia lines. Efficacy of the treatment can be assessed by determining if the subcutaneous echogenicity of the affected area decreased after treatment (as compared to that at baseline), if there is increased contrast at the interface between the subcutaneous fat and the skin, and/or if there is increased visualization or distinctness of the fascia bands under ultrasound. In another example, in certain lipedema patients, efficacy of the treatment can be assessed by determining if the subcutaneous echogenicity of the affected area decreased after treatment (as compared to that at baseline), if there is increased contrast at the interface between the subcutaneous fat and the skin, and/or if there is increased visualization or distinctness of the fascia bands under ultrasound. The methods can comprise maintaining, tapering, reducing, or stopping the administered amount of the acebilustat or other selective LTA4H inhibitor in the therapy if the therapy is determined to be efficacious. The methods can comprise increasing the administered amount of the acebilustat or other selective LTA4H inhibitor in the therapy if it is determined not to be efficacious or likely to be more efficacious if dosing is increased in daily amount or via a change in the administration schedule. Alternatively, the methods can comprise stopping therapy if it is determined not to be efficacious.

For lymphedema, treatment, for example, can commence at any time after the onset of stage 0 lymphedema, e.g., where treatment stabilizes or reverses patient condition to a non-symptomatic state. Treatment with the methods of the invention can, for example, commence following onset of any clinically evident lymphedema, such as stage 1. Treatment with the methods of the invention can also, for example, commence following onset of stage 2 lymphedema. Treatment with the methods of the invention can commence following onset of stage 3 lymphedema. Treatment can also commence before the onset of stage 0 lymphedema but after surgery, radiotherapy or other medical intervention that increases the risk of developing lymphedema.

The swelling that can accompany lymphedema disease progression can be unilateral or bilateral, and may worsen when the weather is warm, before menstruation occurs, following physical exertion, and/or after the limb remains for a long time in a dependent position. It can affect any part of a limb (isolated proximal or distal) or the entire extremity, or the face, head and neck, trunk, breast or genitalia; it can restrict range of motion. Disability and emotional distress can be significant, especially when lymphedema results from medical or surgical treatment. Skin changes are common and include hyperkeratosis, hyperpigmentation, lichenification, verrucae, papillomas, and fungal infections. The methods of the invention include methods to treat any and all of these conditions and symptoms, including but not limited to by administration of acebilustat as described herein.

Lymphangitis or cellulitis can develop, for example, when bacteria traverse the skin barrier, which is impaired in lymphedema. Cellulitis in lymphedema may be characterized by only very subtle changes in the limb, and can be difficult to diagnose or eradicate. Lymphangitis is frequently streptococcal, causing erysipelas; sometimes it is staphylococcal. The affected limb becomes red and feels hot; red streaks may extend proximally from the point of entry, and lymphadenopathy may develop. Rarely, the skin breaks down. Rarely, long-standing lymphedema leads to lymphangiosarcoma (Stewart-Treves syndrome), usually in postmastectomy patients and in patients with filariasis. The methods of the invention include methods to treat any and all of these conditions and symptoms, including but not limited to by administration of acebilustat as described herein. Without treatment, cellular overgrowth, adipose deposition and fibrosis promote the progressive anatomic distortion and loss of function of the affected areas. Additionally, impaired trafficking of antigen-presenting cells in lymph hampers local immune surveillance of the lymphedematous region(s) to the draining lymph nodes. Thus, there is chronic inflammation, infection, and hardening of the skin that, in turn, results in further lymph vessel damage and distortion of the shape of the affected body parts. Moreover, there is a high degree of dysfunction due to physical factors such as a decrease in joint mobility causing reduced amplitude of movements, increased extremity weight, increased pain, and impaired ability to perform day-to-day tasks. The methods of the invention include methods to treat any and all of these conditions and symptoms, including but not limited to by administration of acebilustat or other selective LTA4H inhibitor, as described herein.

Pathological skin changes associated with lymphedema include an increase in cellularity of layers of the skin, accumulation of glycoproteins, loss of elasticity, and subdermal increase in adipose layer. The methods of the invention include methods to treat any and all of these conditions and symptoms, including, but not limited to, by administration of acebilustat or other selective LTA4H inhibitor as described herein.

Those of skill will also thus appreciate that the methods of the invention are applicable to the treatment and prevention of lymphedema including its signs and symptoms such as those associated with the following clinical indicia of lymphedema. A number of clinical indicia can be used to diagnose lymphedema and to monitor the effectiveness of therapy, including treatment with the compositions and methods of the present invention. The invention provides methods of determining efficacy of a lymphedema treatment in a subject in need thereof by (a) measuring an endpoint of a clinical indication in a patient, where the endpoint is measured after treatment has started, (b) comparing the endpoint of the clinical indication to a baseline or reference, where the baseline or reference is measured in the same subject or a similar subject population before treatment is begun, and (c) determining the efficacy of the lymphedema treatment based on the comparison step.

Analysis of clinical indicia may include measurement of dermal thickness; change of lymphedema volume of leg/arm/hand; change of stagnation of fluid at level of shoulder/trunk; change of extracellular fluid in arm; change of thickness and reflectivity of cutis and subcutis of arm/shoulder/trunk; change of elasticity of skin and subcutaneous tissue of arm; change of lymphatic architecture and function; change of venous circulation in arm/trunk; number of episodes of cellulitis.

When imaging is used to diagnose lymphedema or assess disease state or progression, the most common modality for diagnosis is indirect radionuclide lymphoscintigraphy. This procedure requires subcutaneous injection of an appropriate radiolabeled tracer, for example ^{99m}Tc-antimony sulfide colloid or ^{99m}Tc-labeled human serum albumin. Criteria for the diagnosis of lymphatic dysfunction include: (1) delayed, asymmetric or absent visualization of regional lymph nodes; (2) asymmetric visualization of lymphatic channels; (3) collateral lymphatic channels; (4) dermal backflow; (5) interrupted vascular structures; and (6) visualization of the lymph nodes of the deep lymphatic system. The presence of “dermal back-flow” is considered abnormal. It is interpreted to represent the extravasation of lymph fluid from the lymphatics into the interstitium as a result of lymphatic and/or venous hypertension. Beyond lymphoscintigraphy, magnetic resonance imaging and computerized axial tomography have clinical utility. These imaging techniques permit objective documentation of the structural changes caused by lymphedema. Recent advances in the magnetic resonance approach have improved the visualization of lymphatic vascular anomalies in both nonenhanced and contrast-enhanced applications (see, for example, Pankaj et al. (2013) World J Surg Oncol. 2013; 11: 237). As an alternative, bioelectrical impedance has been used to detect and monitor upper limb lymphedema (see Ridner et al. (2009) Lymphat Res Biol. 7(1): 11-15), which uses characteristics of frequency-dependent current flow to quantify changes in extracellular fluid. In various embodiments, such technology is used to monitor the progress of therapy of a patient treated in accordance with the invention or to identify a patient that may benefit from such treatment. Imaging can also include indocyanine green (ICG) fluorescent lymphography as described, for example in Suami et al., BMC Cancer 19, 985 (2019). https://doi.org/10.1186/s12885-019-6192-1, the contents of which are expressly incorporated by reference herein.

The effectiveness of treatment by the methods of the invention will be evidenced by improvement in disease symptoms and pathology. Individuals being treated and medical practitioners may choose to evaluate success, monitor the course of treatment, adjust dosage and timing, etc. by any convenient indicia.

In some embodiments of the invention, e.g., treatment of patients with established disease (e.g., lymphatic dysfunction-associated abnormality of subcutaneous tissue or lymphatic disease of the subcutaneous tissue), improvements in the architecture of the adipose tissue of the affected area provide a convenient method for assessing treatment success. In some embodiments, a treatment provided herein is efficacious if, after a period of time from the onset of treatment (e.g., 2 weeks, 1 month, 2 months, 3 months, 4 months, 5 months, 6 months or longer), there is a decrease in fat deposition or change in the architecture of the adipose tissue (as measured by ultrasound) of at least one affected region as compared to that of the at least one affected region prior to the onset of treatment.

In some embodiments of the invention, e.g., treatment of patients with established lymphedema or lipedema, improvements in the architecture of the skin provide a convenient method for assessing treatment success. For example, in lymphedema, dermal thickness reflects the architectural changes in lymphedema. See, for example, Mellor et al., Breast J 2004; 10:496-503; Hacard et al. Skin Res Technol 2014; 20: 274-81, each herein specifically incorporated by reference. The invention encompasses methods that reduce dermal thickening associated with lymphedema. Dermal thickness can be, for example, measured with factory calibrated skinfold calipers, such as Lange skinfold calipers, Model EQ0014921. In additional aspects, dermal thickness or the decrease in dermal thickening is measured using ultrasound. In some embodiments, a treatment provided herein is efficacious if, after a period of time from the onset of treatment (e.g., 2 weeks, 1 month, 2 months, 3 months, 4 months, 5 months, 6 months or longer), there is a decrease in dermal thickness of at least one affected region (e.g., limb) as compared to the dermal thickness of the at least one affected region prior to the onset of treatment. The decrease in dermal thickness observed with successful treatment can be a decrease of at least about 0.5 mm, about 1 mm, at least about 2.5 mm, at least about 5 mm, at least about 7.5 mm, at least about 10 mm, and may be at least about 12.5 mm, at least about 15 mm, or more, as compared to that before the treatment is initiated (referred to herein as “baseline”) or at an earlier point in the treatment regimen. In yet additional aspects, the dermal thickening or dermal thickness can be decreased by at least about 5%, about 10%, about 15%, about 20%, about 25%, about 30%, about 35%, about 40%, about 45%, or more (for example, at 3 or 6 months after acebilustat treatment), as compared to that before the treatment is initiated (referred to herein as “baseline”) or at an earlier point in the treatment regimen. Additional measurements for determining a correction of pathologies of skin architecture may include, for example, DEXA scanning, direct biopsy, visual inspection, etc. Dermal thickness and architecture, e.g., presence of hyperkeratosis, dermal collagen, and adipose deposition in an affected limb can be monitored. In yet further aspect, the subdermal thickness is reduced by at least about 5%, 10%, 15% or 20%, for example, 3 or 6 months (or less) after acebilustat treatment as compared to baseline. In certain specific aspects, subdermal thickness is reduced by at least about 10%, 15% or 20% after 6 months of treatment with acebilustat as compared to baseline. In additional aspects, subcutaneous thickness is reduced by at least about 5%, 10%, 15% or 20%, for example, 3 or 6 months after acebilustat treatment as compared to baseline. In certain specific aspects, subcutaneous thickness is reduced by at least about 10%, 15% or 20% after 6 months of treatment with acebilustat as compared to baseline.

The ultrasound at baseline and after treatment can be read or assessed by a radiologist or technician, for example. In additional aspects, the reading and analysis of the ultrasound is automated. For example, the ultrasound image(s) can also be read or analyzed by a computer-based system, including, for example, ultrasound imaging software and/or ultrasound reporting software. In yet additional aspects, the ultrasound image(s) are read or assessed by an artificial intelligence (AI) system. In yet further aspects, the ultrasound can be read or assessed by a radiologist or technician as well as an automated system, for example a computer-based system or an AI system. The AI system can, for example, comprise a machine learning program or deep learning. The AI system can include one or more algorithms that are programmed, trained and/or configured to assess the ultrasound image, including, but not limited to, detecting or measuring dermal thickness, subdermal thickness, and/or subcutaneous echogenicity. In certain aspects, the AI system saves the image or measurements from the ultrasound at baseline and compares it to the ultrasound image(s) after treatment. In yet additional aspects, the automated, computer or AI system enhances the image for example by reducing noise and/or by improving contrast. The use of AI in ultrasonography has been described, for example, in Kim 2021, Ultrasonography 40(3): 313-317; and Edwards et al. (2022), Ultrasound 30(4): 273-282; the contents of each of which are expressly incorporated by reference herein.

In yet additional aspects, the ultrasound is an automated or semi-automated ultrasound system that, for example, automatically scans the limb with an ultrasound transducer. In certain aspects, automated or semi-automated ultrasound system comprises a computer-based system or an AI system.

In some embodiments, a change in the volume of the affected limb is measured as a measure of treatment success, i.e., the volume declines with successful treatment of lipedema and lymphedema. Volume can be measured by any of a number of methods in the art, e.g., circumferential measurements, water displacement volumetry, etc. For example, an assessor may use a standardized tape measure for circumference measurements taken every 2-6 cm, and calculating the volume by, for example, the truncated cone method. Successful treatment can reduce, or decrease, the volume of lipedematous body parts or lymphedematous body parts (both the fluid and tissue components). In some instances, volume is decreased about 1.5-fold, about 2-fold or more after treatment, i.e., as compared to the volume before treatment, for example, about 1.5-fold, about 2-fold or more, about 3-fold or more, about 4-fold or more, sometimes about 5-fold or more, about 10-fold or more, about 15-fold or more, in some instances about 20-fold or more, about 50 fold- or more, etc. In other words, the volume is decreased by about 50 milliliters or more, 100 milliliters or more, 200 milliliters or more, 300 milliliters or more, 400 milliliters or more, 500 milliliters or more. In yet additional aspects, volume is decreased by at least about 5%, 10%, 15 or 20%, or more. In some instances, the volume is restored to normal volume, i.e., the volume prior to the onset of the lymphedema, e.g., the volume of the unaffected bilateral tissue.

In some embodiments, the level of serum LTB₄ can be used for diagnosis, or selecting, or stratifying patients for therapy, or for monitoring efficacy of a disease or condition described herein. A reference value of normal LTB₄ can be (depending on assay type and format and individual laboratory practices) up to about 50 μg/mL or greater, up to about 100 μg/mL or greater, up to about 200 μg/mL or greater, up to about 250 μg/mL or greater, up to about 300 μg/mL. Individuals with lymphedema may have elevated baseline levels of serum LTB₄, where the serum levels are up to about 1000 μg/ml or greater, and can range from about 500 μg/ml to about 1500 μg/ml. In certain aspects, the level of serum LTB4 in a sample obtained from a patient is measured and if it above a predetermined threshold level, the patient is administered acebilustat or other selective LTA4H inhibitor. The treatment provided in the invention can be determined to be efficacious if, after treatment has started, the endpoint LTB₄ level of the subject decreases from the baseline LTB₄ level. In other embodiments, the treatment provided in the invention is efficacious if, after treatment has started, the endpoint LTB₄ level is lower than the baseline LTB₄ level by about 2-fold or more, about 3-fold or more, about 4-fold or more, about 5-fold or more, about 10-fold or more, about 15-fold or more. For example, in some embodiments a higher or elevated level of LTB₄ in a blood sample, of at least about 3-fold higher, at least about 4-fold higher, at least about 5-fold, at least about 10-fold higher, at least about 15-fold higher than the control value indicates that the patient is in need of treatment using a therapy of the invention or that the patient is likely to respond to a therapy of the invention. Standard methods for assessing LTB₄ levels are utilized. The method further comprises administering an effective amount of acebilustat or other selective LTA4H inhibitors to a patient determined to be likely to benefit from, in need of, or likely to respond to, a therapy of the invention, thereby treating or preventing lymphedema in the patient.

As used therein, a “therapeutically effective amount” or an “effective amount” refers to that amount of a compound or drug that, when administered to a mammal, preferably a human, is sufficient to effect treatment, as defined below, of a disease or condition of interest in the mammal, preferably a human. The amount of a compound of the invention which constitutes a “therapeutically effective amount” or an “effective amount” will vary depending on, for example, the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy, but it can be determined routinely by one of ordinary skill in the art having regard to his own knowledge and to this disclosure. In certain aspects, an effective amount or a therapeutically effective amount of acebilustat or other selective LTA4H inhibitor is an amount that inhibits LTA4H or inhibits LTB4, and/or that treats or inhibits or decreases the severity of the disease (lipedema or lymphedema) and/or inhibits or decreases the severity of lymphatic dysfunction-associated abnormality of subcutaneous tissue or lymphatic disease of the subcutaneous tissue and/or reduces and/or improves skin turgor, histology and/or function or increases lymphatic flow and/or improves vascular function and/or reduces adipose tissue and/or results ultrasonographic changes.

“Treating” or “treatment” as used herein covers the treatment of lipedema, lymphedema, or lymphatic dysfunction-associated abnormality of subcutaneous tissue or lymphatic disease of the subcutaneous tissue, preferably in a human, and includes, for example: (i) inhibiting or decreasing the severity of the disease or condition, or one or more symptoms thereof, i.e., arresting or slowing development or progression of the disease or condition inhibits or decreases the severity of lymphatic dysfunction-associated abnormality of subcutaneous tissue or lymphatic disease of the subcutaneous tissue and/or reduces and/or improves skin turgor, histology and/or function or increases lymphatic flow and/or improves vascular function and/or reduces adipose tissue and/or results ultrasonographic changes, and/or ameliorating or decreasing one or more symptoms; (ii) relieving the disease or condition, i.e., causing regression of the disease or condition, or one more symptoms thereof, and/or (iii) stabilizing the disease or condition. “Treating” or “treatment” can include decreasing the risk of progression of lipedema, lymphedema, and/or lymphatic dysfunction-associated abnormality of subcutaneous tissue or lymphatic disease of the subcutaneous tissue.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound described herein, for example, acebilustat or other selective LTA4H inhibitor and/or an additional therapeutic agent, and a medium generally accepted in the art for the delivery of the biologically active compound to mammals, for example, humans. Such a medium includes all pharmaceutically acceptable carriers, diluents or excipients.

“Optional” or “optionally” means that the subsequently described event or circumstances may or may not occur, and that the description includes instances where said event or circumstance occurs and instances in which it does not.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which, for example, has been approved by the United States Food and Drug Administration as being acceptable for use in humans or domestic animals.

Administration of the compounds or drugs described herein encompasses administration of a pharmaceutically acceptable salt of said compound or drug, for example, administration of a pharmaceutically acceptable salt of acebilustat or other selective LTA4H inhibitor. Administration of the compounds or drugs as described herein (such as acebilustat or other additional therapeutic agent), or their pharmaceutically acceptable salts, in pure form or in an appropriate pharmaceutical composition, can be carried out via any of the accepted modes of administration of agents for serving similar utilities. As described herein, an exemplary mode of administration for acebilustat is oral administration. The pharmaceutical compositions described herein can be prepared by combining a compound or drug with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols.

Routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20^{th} Edition (Philadelphia College of Pharmacy and Science, 2000). The composition to be administered will, in any event, contain a therapeutically effective amount of the compound or drug, or a pharmaceutically acceptable salt thereof, for treatment of a disease or condition of interest in accordance with the teachings of this invention.

As described above, the pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Formulations suitable for enteral administration, such as, for example, administration topically (e.g., as solutions, lotions, creams, paste, emulsions, suspensions, etc.), orally, rectally, vaginally, or by inhalation, include capsules, liquid solutions, emulsions, suspensions, and elixirs. For example, if prepared for topical applications, the compositions may comprise a biocompatible organic solvent, e.g., an isopropyl ester such as isopropyl myristate and isopropyl palmitate; a polar lipid, e.g., lecithin, phosphatidylcholine, etc., a surfactant, e.g., docusate sodium, docusate sodium benzoate, docusate calcium, tween 80, polysorbate 80; water; and/or urea (present at a concentration of about 5 to 20% by mass of the final composition). In some instances, a topical formulation will comprise an enhancer for skin penetration, such as SEPA 09. Examples of topical formulations may be found in, e.g., U.S. Pat. Nos. 5,654,337, 5,093,133, 5,210,099, 3,957,971, 5,016,652, the complete disclosures of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

A pharmaceutical composition can be in the form of a solid or liquid. In one aspect, the carrier(s) are particulate, so that the compositions are, for example, in tablet or powder form. In one aspect, the composition can be an encapsulated powder or granular form. In another aspect, an encapsulated powder or granular formulation can be opened and sprinkled in food or administered by gastric intubation. The carrier(s) can be liquid, with the compositions being, for example, an oral syrup, injectable liquid or an aerosol, which is useful in, for example, inhalatory administration. When intended for oral administration, the pharmaceutical composition can be in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition can be in the form of a liquid, for example, an elixir, syrup, solution, emulsion or suspension. The liquid can be for oral administration or for delivery by injection, as two examples. When intended for oral administration, a composition can contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether solutions, suspensions or other like form, can include one or more of the following adjuvants: sterile diluents such as water for injection, saline solution, preferably physiological saline, Ringer's solution, isotonic sodium chloride or physiological saline, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The invention is illustrated by the following non-limiting examples.

### EXAMPLES

### Example 1: Ultrasonographic Skin Changes after Early Therapeutic LTB4 Antagonism in Lymphedema

Lymphedema is a chronic disease characterized by the accumulation of excess fluid in the interstitial spaces, leading to regional edema. Currently, there are no cures for this debilitating disease. Lymphedema pathophysiology involves pro-inflammatory mediators such as leukotriene B4 (LTB4). Dermal thickness and subcutaneous echogenicity are increased in chronic lymphedema. This study explores the use of acebilustat, an inhibitor of leukotriene A4 hydrolase (LTA4H), the rate limiting step in leukotriene B4 (LTB4) synthesis. Published work in animal models supports LTB4 antagonism to reverse lymphedema and associated tissue changes.

HEAL is an ongoing single center, Phase 2 trial of acebilustat in human upper limb lymphedema. We present the early ultrasonographic responses of a participant who has completed 3 months of a total 6-month exposure. The primary endpoint for this trial involves quantitative analysis of dermal ultrasound skin changes.

Profound ultrasonographic forearm skin changes in the subject treated with acebilustat for three months were observed. Specifically, dermal ultrasound of the forearm of the subject with Stage II lymphedema three months after treatment showed reduced dermal thickness and reduced echogenicity as compared to baseline. In addition, there was enhanced demarcation of the dermo-epidermal junction and subcutaneous fascial bands. Subcutaneous tissue echogenicity was also decreased.

This case report demonstrates evidence of profound ultrasonographic skin changes in the upper limb of this partially treated subject. Acebilustat reduced dermal thickness and subcutaneous echogenicity in upper limb lymphedema patients. Reduced dermal thickness and subcutaneous echogenicity suggest acebilustat may target inflammation and excess water content of affected tissues, replicating responses previously observed in pre-clinical models. These preliminary results lend great promise to the HEAL trial.

### Example 2: Early Data in Expanded Access Program (EAP) Patients Treating with Acebilustat for Lymphedema

The main arm of the study is the HEAL clinical trial (NCT05203835), described above and in WO2023107608 (the contents of which are expressly incorporated by reference herein, was designed to investigate the response of unilateral upper extremity (arm) lymphedema, during pharmacologic treatment of lymphedema with oral placebo and oral acebilustat. This example describes initial data from the expanded access program which includes patients with lymphedema of any non-cancer cause, patients with genetic and vascular conditions, as well as patients with lipedema and obesity.

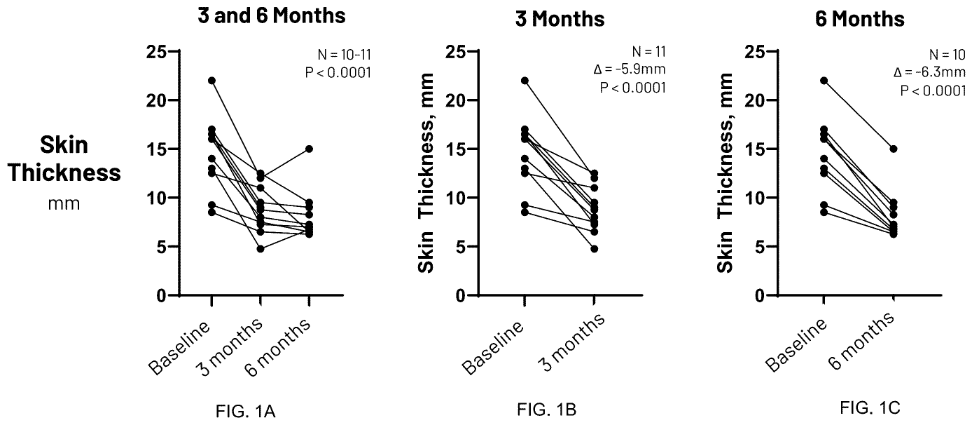

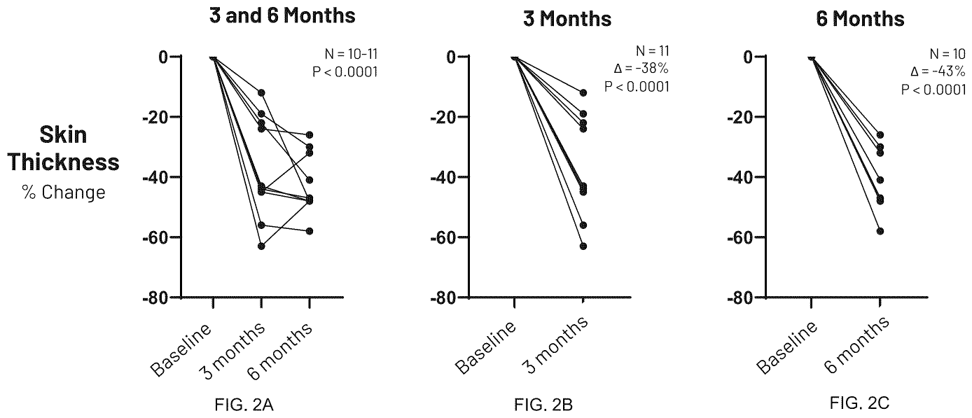

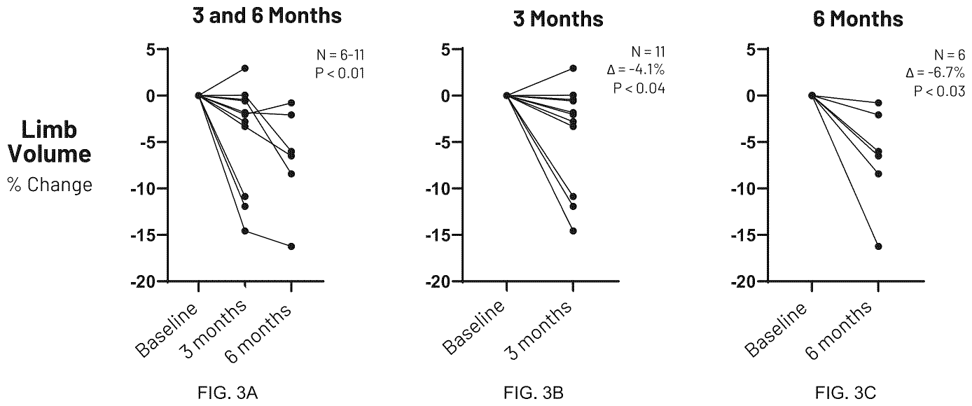

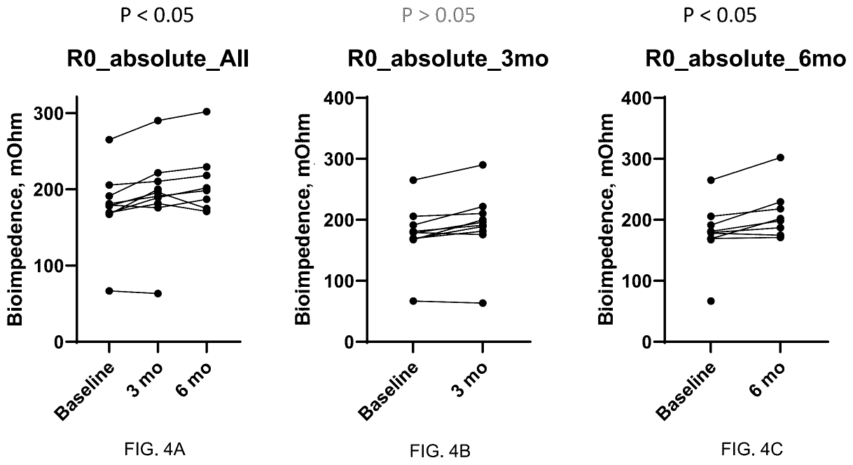

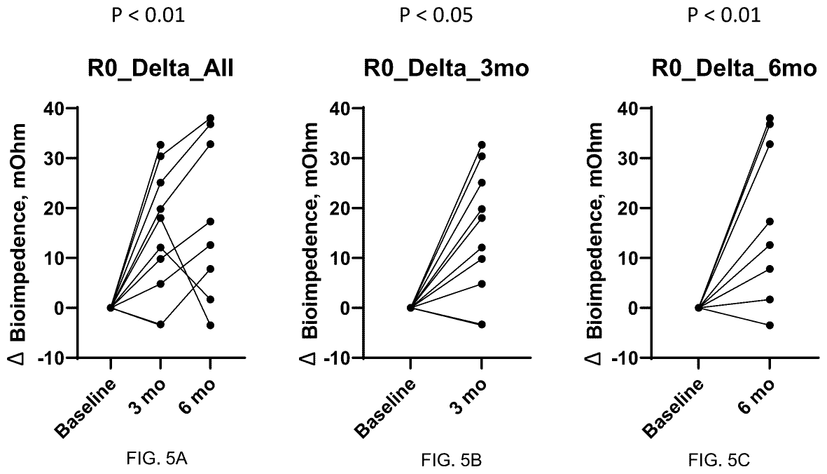

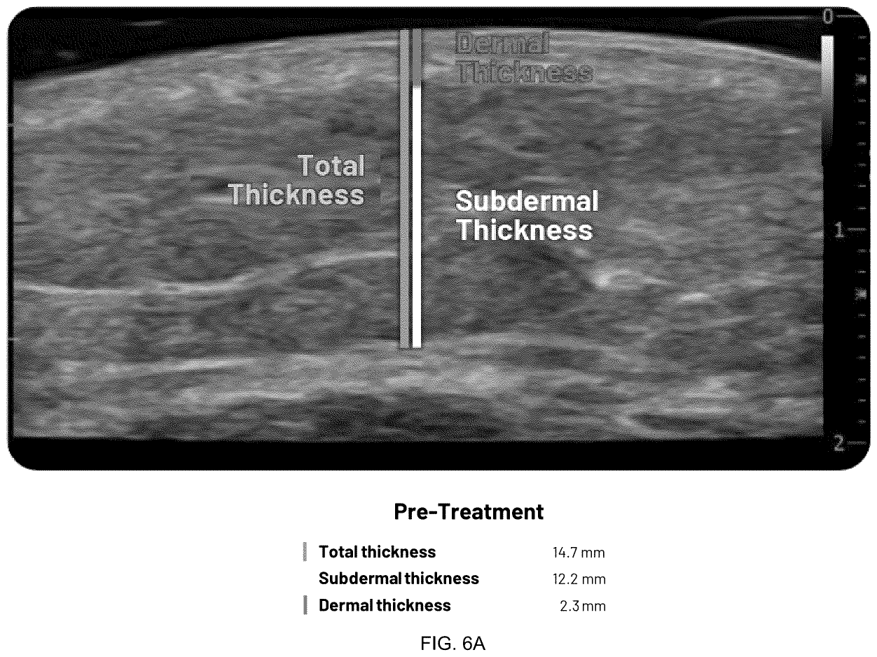

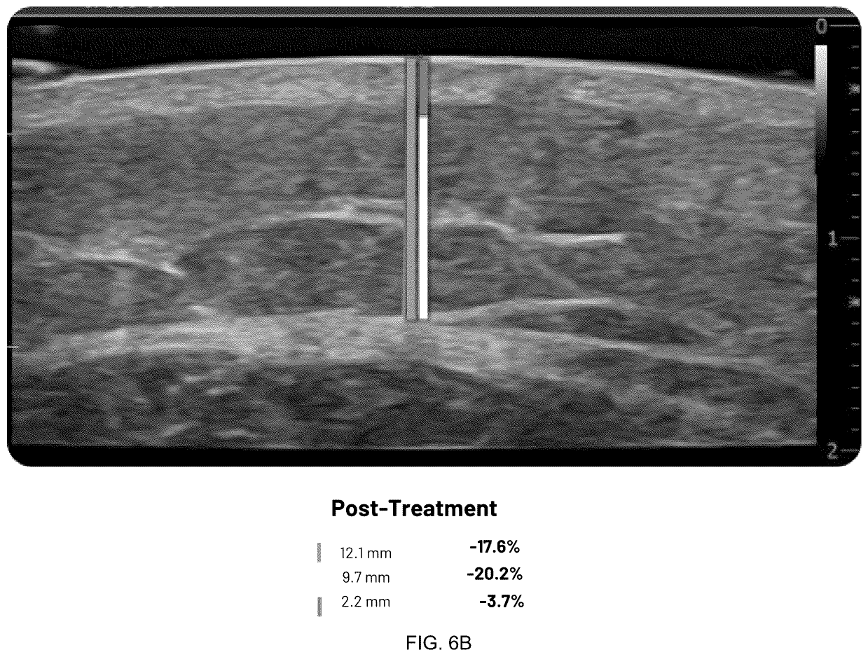

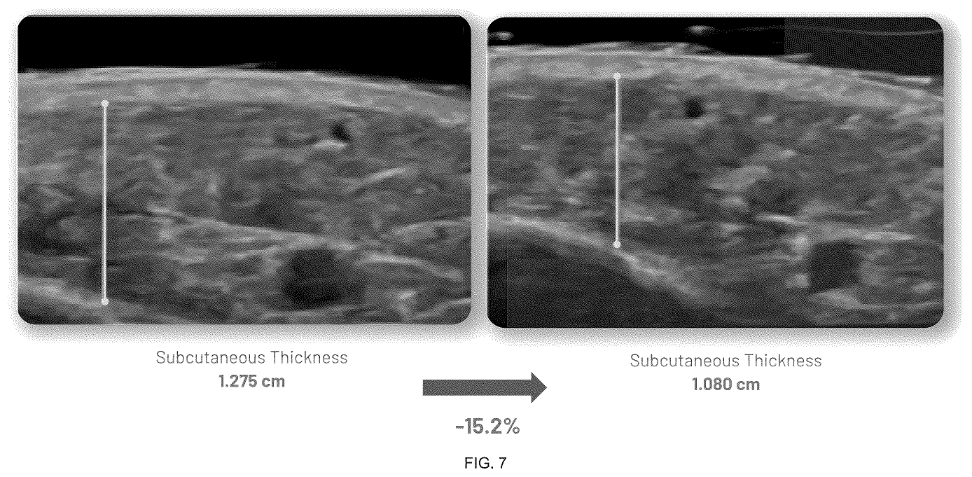

FIGS. 1A-1C and 2A-2C show that skin thickness was dramatically reduced after 3 and 6 months of treatment with acebilustat. FIG. 2C shows that a 43% reduction in skin thickness was observed after 6 months of treatment with acebilustat. FIG. 3C shows that there was 6.7% reduction in limb volume after 6 months and FIG. 5C shows that a significantly lower water content in the affected limb was observed after 6 months of treatment with acebilustat. FIGS. 6A-6B are dermal ultrasound images of a patient with right leg lymphedema prior to treatment (FIG. 6A) and 3 months after treatment with acebilustat. Subdermal thickness was reduced by over 20.2%, dermal thickness was reduced by 3.7%, and total thickness was reduced by 17.6% at 6 months. Lastly, FIG. 7 shows (in a different patient than that of FIGS. 6A and 6B) that a 15.2% reduction in subcutaneous thickness was observed after 6 months. Overall, these results demonstrate profound changes in skin thickness, subdermal thickness and the in the subcutaneous space in lymphedema patients after treatment with acebilustat.

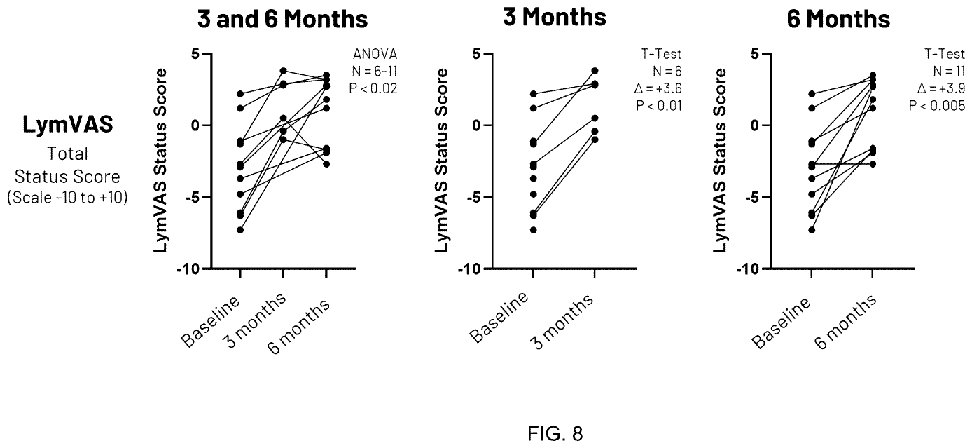

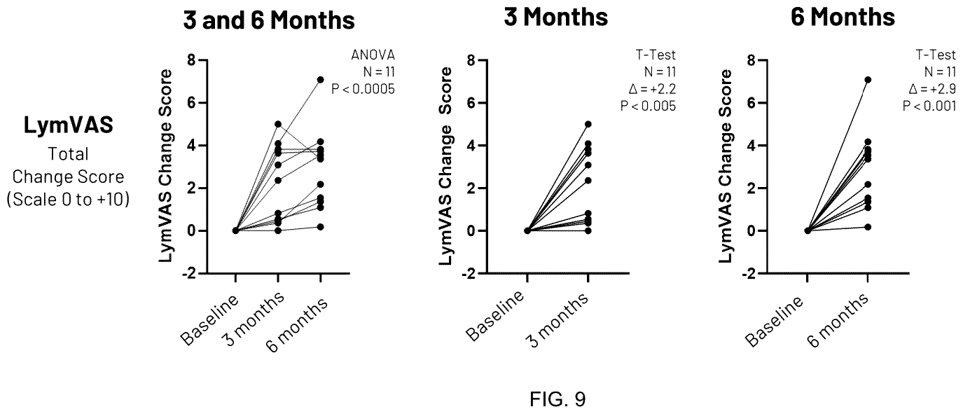

Quality of life indicators were also evaluated in the 11 EAP patients using LymVAS, a tool to interrogate lymphedema patient experience and is based on experience and practice from prior lymphedema trials. LymVAS uses a sliding visual analog scale, wherein patients move the slider along a line to indicate the degree of response to a question. LymVAS has two segments: 1. Status Domain measure the state of the patient in the moment (Questions are asked in the following manner: “How is ______ today?” and 2. Change (or delta) Domain measures change in patient's experience after starting treatment: (Questions are asked in the following manner: “How has ______ changed since starting treatment?” FIGS. 8 and 9 show that there was a significant improvement in LymVAS quality of life Status Score and Change Score at 3 months after acebilustat treatment and that that improvement was maintained 6 months after treatment.

### Example 3: Treatment of Expanded Access Program (EAP) Patients with Acebilustat for Lipedema

The objectives of this expanded access study are to provide early access to treatment with acebilustat to individuals with lipedema resistant to conventional therapy and to evaluate the safety of acebilustat in lipedema patients. Patients are included in the study if they have a clinical diagnosis of lymphedema for >6 months. Patients are excluded if they have upper extremity lymphedema or currently active cancer or a history of cancer. Acebilustat is administered at 100 mg once daily for 12 months. The primary outcome to be studied is change in integumentary thickness and composition of affected areas by ultrasonography. Secondary outcomes include change in skin fold thickness by caliper, change in limb volume, quantitative changes in subdermal tissue thickness and architecture by ultrasonography, quality of life, and pain.

### REFERENCES

1. Iker et al. 2019, Characterizing Lower Extremity Lymphedema and Lipedema with Cutaneous Ultrasonography and an Objective Computer-Assisted Measurement of Dermal Echogenicity Lymphatic Research and Biology, 17(5): 525-530.
2. Herbst et al. (2021), Standard of care for lipedema in the United States, Phlebology 36(10): 779-796.
3. Michelini et al. 2020, Aldo-Keto Reductase 1C1 (AKR1C1) as the First Mutated Gene in a Family with Nonsyndromic Primary Lipedema, Int J Mol Sci 21(17): 6264.
4. Bilancini S, Lucchi M, Tucci S, Eleuteri P. Functional lymphatic alterations in patients suffering from lipedema. Angiology. 1995; 46(4):333-339.
5. Rockson SG and Rivera KK. Estimating the population burden of lymphedema. Ann NY Acad Sci. 2008; 1131:147-54.
6. Mortimer PS and Rockson SG. New developments in clinical aspects of lymphatic disease. J Clin Invest. 2014; 124:915-21.
7. Rockson SG, Keeley V, Kilbreath S, Szuba A and Towers A. Cancer-associated secondary lymphoedema. Nat Rev Dis Primers. 2019; 5:22.
8. Suehiro et al. (2013), Significance of Ultrasound Examination of Skin and Subcutaneous Tissue in Secondary Lower Extremity Lymphedema, Ann Vasc Dis 6(2): 180-188.
9. Vyas et al. (2023), Lipedema, www.ncbi.nlm.nih.gov/books/NBK573066/; accessed on Jun. 5, 2023.
10. Wolf et al. (2022), A distinct M2 macrophage infiltrate and transcriptomic profile decisively influence adipocyte differentiation in lipedema, Front. Immunol. 13 doi.org/10.3389/fimmu.2022.1004609.
11. Ihnatsenka et al. (2010), Ultrasound: Basic understanding and learning the language, Int J Shoulder Surg 4(3): 55-62.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. A method of treating lipedema in a patient in need thereof comprising administering to said patient a therapeutically effective amount of acebilustat.

2-7. (canceled)

8. The method of claim 1, wherein the lipedema is Stage I lipedema.

9. The method of claim 1, wherein the lipedema is Stage II lipedema.

10. The method of claim 1, wherein the lipedema is Stage III lipedema.

11. The method of claim 1, wherein the lipedema is Stage IV lipedema (lipo-lymphedema).

12. The method of claim 1, wherein the treatment results in a decrease in adipose tissue in the affected area.

13-30. (canceled)

31. A method of treating a lymphatic dysfunction-associated abnormality of subcutaneous tissue or lymphatic disease of the subcutaneous tissue in a patient in need thereof comprising administering to said patient a therapeutically effective amount of acebilustat.

32. The method of claim 31, wherein the subcutaneous tissue is subcutaneous adipose tissue (SAT).

33. The method of claim 31, wherein the subcutaneous tissue is fibrotic tissue.

34. The method of claim 32, wherein the abnormality is selected from the group consisting of an accumulation or deposition of SAT, inflammation of the SAT, and a disruption of the fascia bands of the SAT, or a combination thereof.

35. The method of claim 32, wherein visualization by ultrasound of the fascia bands in the affected SAT is improved after treatment.

36. The method of claim 31, wherein the patient is suffering from lymphedema.

37. The method of claim 36, wherein the lymphedema is primary lymphedema.

38. The method of claim 36, wherein the lymphedema is secondary lymphedema.

39. The method of claim 36, wherein the lymphedema is cryptogenic lymphedema.

40. The method of claim 36, wherein the lymphedema is upper limb lymphedema.

41. The method of claim 36, wherein the lymphedema is lower limb lymphedema.

42. The method of claim 36, wherein the lymphedema is head, neck and trunk lymphedema.

43-45. (canceled)

46. The method of claim 38, wherein the patient has previously undergone surgery and/or radiation therapy for cancer.

47. (canceled)

48. The method of claim 31, wherein the subdermal thickness of the affected area is reduced by at least about 10% after administration of acebilustat for three months or less, as compared to baseline.

49. The method of claim 48, wherein the subdermal thickness is reduced by at least about 20% after administration of acebilustat for three months or less.

50. The method of claim 31, wherein the subcutaneous thickness of the affected area is reduced by at least about 10% after administration of acebilustat for six months or less, as compared to baseline.

51. The method of claim 50, wherein the subcutaneous thickness of the affected area is reduced by at least about 10% after administration of acebilustat for three months or less, as compared to baseline.

52. The method of claim 31, wherein the patient is suffering from lipedema.

53-55. (canceled)

56. The method of claim 31, wherein the acebilustat is administered orally.

57. The method of claim 56, wherein the daily dose of acebilustat is about 200 mg/day or less.

58-75. (canceled)

76. A method of preventing or reducing the risk of a lymphatic dysfunction-associated abnormality of subcutaneous tissue or lymphatic disease of the subcutaneous tissue in a patient at risk thereof, comprising administering to said patient a therapeutically effective amount of acebilustat.

77. The method of claim 76, wherein the method prevents or reduces the risk of lymphedema.

78-81. (canceled)

82. A method of reducing the subdermal thickness of a lymphedematous area in a patient suffering from lymphedema, the method comprising administering to said patient a therapeutically effective amount of acebilustat, wherein the subdermal thickness of the lymphedematous area is reduced by at least about 10% after the administration of acebilustat for six months or less, as compared to baseline.

83. The method of claim 82, wherein the subdermal thickness is reduced by at least about 10% after the administration of acebilustat for three months or less.

84-89. (canceled)
